(19) United States
(12) Patent Application Publication
(10) Pub. No.: US 2022/0118458 A1
(43) Pub. Date: Apr. 21, 2022

(54) HEAT TREATED PULSE FLOURS

(71) Applicant: Eat JUST, Inc., San Francisco, CA (US)

(72) Inventors: Michael JAMROS, Oakland, CA (US); Sayani MALLICK, Emeryville, CA (US); Vivek SHARMA, Fremont, CA (US); Nathaniel PARK, Alameda, CA (US); Elankovan PONNAMPALAM, Milpitas, CA (US)

(21) Appl. No.: 17/506,126

(22) Filed: Oct. 20, 2021

Related U.S. Application Data
(60) Provisional application No. 63/094,185, filed on Oct. 20, 2020.

(51) Int. Cl. B02B 1/08; A23J 1/14; A23L 15/00; A23L 33/185; B01D 61/14; B01D 61/16; C08L 99/00; C08H 99/00

(52) U.S. Cl. CPC B02B 1/08; A23J 1/14; A23L 15/35; A23L 15/20; A23L 33/185; B01D 2311/04; B01D 61/16; C08L 99/00; C08H 99/00; A23V 2002/00; B01D 61/145

(57) ABSTRACT

Heat treated pulse flour, pulse protein isolates obtained from heat treated pulse flour, food compositions containing such isolates, and methods for preparing heat treated pulse flours and pulse protein isolates are disclosed. The amount of volatile small molecule compounds present in the heat treated pulse flour are decreased or increased.

### CROSS-REFERENCE TO RELATED APPLICATION

This application claims priority from U.S. provisional patent application No. 63/094,185 filed on Oct. 20, 2020. The entire contents of this earlier filed application are hereby incorporated by reference in their entirety.

### FIELD OF THE INVENTION

The present disclosure relates to heat treated pulse flours. The volatile small molecule compounds that are present in the heat treated pulse flour are altered by the heat treatment. Proteins are isolated from the heat treated pulse flours, and the isolated proteins can be used in foods.

### BACKGROUND

Use of plant-based proteins such as soy and pea as animal protein substitutes have garnered increasing attention as consumers seek alternatives to conventional animal-based products to reduce the environmental impacts of animal husbandry and to improve dietary options that minimize the negative implications of consuming many animal protein products.

Conventional methods and processes used for extracting plant protein isolates and concentrates include alkaline extraction, acid precipitation, and filtration methods, including ultrafiltration. The quality of the plant protein compositions produced by these methods is directly dependent on the operating conditions used to prepare them. Typically, plant proteins are isolated from flours prepared from plant material such as pulses. Application of an acidic, alkaline, pH neutral extraction process or filtration methods influences functional properties, e.g., the gelling, foaming or emulsifying properties of the protein compositions obtained, which can make the resulting protein compositions unsuitable for certain applications as well as the taste of the protein isolate. There remains a need for processes of isolating plant-based proteins with physical characteristics and organoleptic properties desirable for the production of food products, including alternatives to conventional products containing animal proteins.

### SUMMARY

In one aspect, the present disclosure provides a method for preparing a heat treated pulse flour. A dehulled pulse or an pulse that is not dehulled (unhulled) is heat treated at one or more desired temperatures, and the heat treated pulse is milled to produce the heat treated pulse flour. In an embodiment, the heat treated pulse flour comprises volatile small molecule compounds, wherein the amount of the volatile small molecule compounds present in the heat treated pulse flour is increased or decreased as compared to the amount of volatile small molecule compounds present in a pulse flour that has not been heat treated. The change in the amount of the volatile small molecule compounds alters the flavor of the pulse flour, the proteins isolated from the pulse flour or the starches and fibers isolated from the pulse flour.

In one embodiment, the heat treatment of the pulse is performed with exposure to steam or without exposure to steam.

In an embodiment, proteins are isolated from the heat treated pulse flour to produce a protein isolate. In one embodiment, extracting proteins from a heat treated milled composition (flour) comprises incubation in an aqueous solution at a pH of from about 1 to about 9 to produce a protein rich fraction containing extracted pulse proteins; applying the protein rich fraction to an ultrafiltration process comprising a semi-permeable membrane to separate a retentate fraction from a permeate fraction based on molecular size at a temperature of from 2° C. to 60° C.; and collecting the retentate fraction containing the pulse protein isolate. In another embodiment, proteins can be extracted from the heat treated pulse flour by isoelectric precipitation (IEP). IEP can be performed by the methods taught in the applicant's patent application WO2017/143298, herein incorporated by reference.

In one embodiment, the proteins are isolated from pulse flour by ultrafiltration (UF). In one embodiment, the proteins are isolated from pulse flour by the methods taught in the applicant's patent applications 62/981,890; 63/018,692; and PCT/US2021/19931 (filed on Feb. 26, 2021), herein incorporated by reference.

In one embodiment, the milled composition is air classified to separate denser flour particles from the less dense particles to prepare air-classified flour, prior to the aqueous extraction step for producing the protein rich fraction containing extracted pulse proteins.

In any embodiments disclosed herein, the milled composition may comprise dry beans, lentils, faba beans, dry peas, chickpeas, cowpeas, bambara beans, pigeon peas, lupins, vetches, adzuki, common beans, fenugreek, long beans, lima beans, runner beans, tepary beans, soy beans, or mucuna beans. In any embodiments of the methods, the milled composition may comprise Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius. In some cases, the milled composition comprises mung beans (Vigna radiata). In other embodiments, the milled composition may comprise almonds and other nuts, seeds such as sesame seeds, sunflower seeds, and other commonly consumed nuts, fruits and seeds.

In any embodiments, the pulse proteins are not precipitated from the protein rich fraction at a pH of from 4 to 6 or 5 to 6.

In any embodiments provided herein, the retentate fraction of the UF prepared protein comprises pulse proteins having a molecular size of less than 100 kilodaltons (kDa). In some cases, the retentate fraction comprises pulse proteins having a molecular size of less than 50 kDa. In some cases, the retentate fraction comprises pulse proteins having a molecular size of less than 25 kDa. In some cases, the retentate fraction comprises pulse proteins having a molecular size of less than 15 kDa.

In any embodiments of the methods, the permeable membrane for UF protein production may be a polymeric membrane, a ceramic membrane, or a metallic membrane. In various embodiments, the permeable membrane is made from polyvinylidine fluoride (PVDF), polyether sulfone (PES), polyacrylonitrile (PAN), polytetrafluoroethylene (PTFE), polyamide-imide (PAI), a natural polymer, rubber, wool, cellulose, stainless steel, tungsten, palladium, an oxide, a nitride, a metallic carbide, aluminum carbide, titanium carbide, or a hydrated aluminosilicate mineral containing an alkali and alkaline-earth metal.

In any embodiment of the methods, the ultrafiltration process is performed at a pressure of from about 20 to about 500 psig.

In another aspect, the present disclosure provides a pulse protein isolate prepared by any one of the methods discussed above or herein.

In another aspect, the present disclosure provides a food composition comprising a pulse protein isolate discussed above or herein, and one or more edible ingredients.

In any of the various embodiments of the isolated pulse protein, the pulse protein may have been isolated from dry beans, lentils, faba beans, dry peas, chickpeas, cowpeas, bambara beans, pigeon peas, lupins, vetches, adzuki, common beans, fenugreek, long beans, lima beans, runner beans, tepary beans, soy beans, or mucuna beans. In any of the various embodiments of the isolated pulse protein, the pulse protein may be isolated from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius. In some cases, the pulse protein is isolated from mung beans (Vigna radiata). In other embodiments, the milled composition may comprise almonds and other nuts, seeds such as sesame seeds, sunflower seeds, and other commonly consumed nuts, fruits and seeds.

In any of the various embodiments of the isolated pulse protein, the pulse protein may include proteins having a molecular size of less than 100 kDa. In some embodiments, the pulse protein includes proteins having a molecular size of less than 50 kDa. In some embodiments, the pulse protein includes proteins having a molecular size of less than 25 kDa. In some embodiments, the pulse protein includes proteins having a molecular size of less than 15 kDa. In some embodiments, the pulse protein includes proteins having a molecular size of from 1 kDa to 99 kDa.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges and all intermediate values are encompassed within the scope of the present disclosure.

In one embodiment, the volatile small molecule compound present in the pulse flour is selected from the group consisting of heptane; 3-methyl, 1-butanol; 4-methyl heptane; 1-pentanol; 2,4-dimethylhept-1-ene; hexanal; benzoic acid, methyl ester; decane; 2-methyl; 1-pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; β-ionone; undecanal; methyleugenol, 3-carene; dodecane; or combinations thereof.

In an embodiment, the amount of volatile small molecule compounds present in the heat treated pulse flour is decreased as compared to the amount of small molecule compounds present in a pulse flour that is not heat treated.

In one embodiment, the amount of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; β-ionone; undecanal; methyleugenol; or combinations thereof present in the heat treated pulse flour is decreased as compared to the amount of small molecule compounds present in a pulse flour that is not heat treated.

In an embodiment, the amount of volatile small molecule compounds present in the heat treated pulse flour is increased as compared to the amount of small molecule compounds present in a pulse flour that is not heat treated.

In one embodiment, the amount of 3-carene or dodecane present in the heat treated pulse flour is increased as compared to the amount of, 3-carene or dodecane present in a pulse flour that is not heat treated.

In one embodiment, the amount of 2,4-dimethylhept-1-ene; decane or benzoic acid, methyl ester present in the heat treated pulse flour remains the same as compared to the amount of 2,4-dimethylhept-1-ene; decane or benzoic acid, methyl ester present in a pulse flour that is not heat treated.

In one embodiment, the amount of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; β-ionone; undecanal; or methyleugenol is decreased while concurrently the amount of 3-carene or dodecane is increased and/or concurrently the amount of 2,4-dimethylhept-1-ene; decane or benzoic acid; methyl ester; or combinations thereof remains the same compared to pulse flour that has not been heat treated.

In one embodiment, the amount of 2,4-dimethylhept-1-ene; benzoic acid, methyl ester; or decane present when the pulse is treated with dry heat (without steam) does not decrease when compared to pulse flour made from unroasted pulses.

In one embodiment, the amount of 2,4-dimethylhept-1-ene; benzoic acid, methyl ester; or decane present when the pulse is treated with steam heat decreases when compared to pulse flour made from unroasted pulses.

In an embodiment, the amount of volatile small molecule compounds present in the heat treated pulse flour are determined by analyzing the volatile small molecule compounds obtained by headspace gas analysis, in-tube extraction dynamic headspace (ITEX-DHS), stirbar sorptive extraction (SBSE), solid phase microextraction (SPME) or purge and trap.

In an embodiment, headspace gas analysis is performed by analyzing the gas phase or vapor portion of a sample in a sealed chromatography vial. A sample to be analyzed is sealed in a chromatography vial, then the vial is heated for a period of time, with or without agitation, allowing the volatile small molecule compounds in the sample to volatilize into the headspace of the chromatography vial. A sample of the headspace gas is then removed by a syringe and analyzed, typically by injection into a GC or GC/MS instrument.

In an embodiment, the volatile small molecule compounds can also be extracted by the purge and trap technique. In the purge and trap technique, a measured amount of a sample is placed in a sealed vessel, then the sample is purged with an inert gas, causing the analyte volatile small molecule compounds to be swept out of the sample. The analytes are then passed over an adsorbent or absorbent surface, which serves as trap where volatile small molecules are retained. The analytes are then desorbed by heating the trap and injected into a GC. GC/MS or other analytical instrument, by backflushing the trap with the carrier gas into, for example, the GC/MS.

In an embodiment, the volatile small molecule compounds can be extracted by ITEX-DHS. Analyte extraction by ITEX-DHS involves repeatedly pumping a syringe inserted into the headspace area of a vial, typically after the sample undergoes an incubation period with heating and agitation, to enrich an adsorbent or absorbent surface within the syringe to which volatile analyte compounds reversibly bind. Next, the adsorbent or absorbent (from the syringe) is heated, which results in desorption of the volatile organic compounds from the adsorbant or absorbant. The desorbed analytes are analyzed by analytical techniques, for example by GC/MS or other chromatographic and/or mass spectroscopic analysis.

In an embodiment, the volatile small molecule compounds can be extracted by stir bar sorptive extraction (SBSE). SBSE is a sample extraction and enrichment technique whereby a magnetic stir bar coated with a sorptive material is introduced into a sample and is used to mix the sample of interest. While the sorptive coated stir bar is in contact with the sample, the analyte volatile compounds bind the stir bar. After a desired incubation time, the analytes adsorbed (or absorbed) onto the stir bar are desorbed from the sorptive material by exposure to heat, solvents or other well understood methods. The desorbed analyte volatile molecule compounds are the analyzed by analytical techniques, for example by GC/MS or other chromatographic and/or mass spectroscopic analysis.

In an embodiment, the volatile small molecule compounds can be extracted by solid phase microextraction (SPME), a solventless sample extraction technique. In SPME, analytes first establish an equilibrium amongst the sample, the headspace of a vial containing the sample, and a polymer-coated fused fiber. The analytes are obtained through the absorption or adsorption (dependent on the fiber) of analyte compounds from the sample onto the fiber, which then transfers analytes into the headspace. Analyte compounds are then introduced to the GC/MS or other analytical instruments, either through via an injection taken from the headspace or the fiber may be inserted directly into the GC/MS for desorption and analysis.

In one embodiment, the proteins isolated from the heat treated pulse flour comprise volatile small molecule compounds, wherein the amount of volatile small molecule compounds present in the heat treated pulse flour is increased or decreased as compared to the amount of small molecule compounds present in a pulse flour that is not heat treated.

In an embodiment, the amount of volatile small molecule compounds present in the isolated protein is decreased as compared to the amount of small molecule compounds present in a pulse flour that is not heat treated.

In one embodiment, the amount of a compound selected from the group consisting of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; or di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; β-ionone; undecanal; methyleugenol; or combinations thereof present in the isolated protein prepared from a heat treated pulse flour is decreased as compared to the amount of small molecule compounds present in an isolated protein prepared from a pulse flour that is not heat treated.

In an embodiment, the amount of volatile a small molecule compound present in the isolated protein prepared from a heat treated flour is increased as compared to the amount of small molecule compounds present in an isolated protein prepared from a pulse flour that is not heat treated.

In one embodiment, the amount of a compound selected from the group consisting of 3-carene and dodecane present in the isolated protein prepared from a heat treated pulse flour is increased as compared to the amount of 3-carene or dodecane present in the isolated protein obtained from pulse flour that is not heat treated.

In one embodiment, the amount of 2,4-dimethylhept-1-ene; decane or benzoic acid, methyl ester present in the isolated protein prepared from a heat treated pulse flour remains the same as compared to the amount of 2,4-dimethylhept-1-ene; decane or benzoic acid, methyl ester present in an isolate protein prepared from pulse flour that is not heat treated.

In one embodiment, the amount of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; β-ionone; undecanal; or methyleugenol are decreased while concurrently the amount of 3-carene or dodecane is increased and/or concurrently, the amount of 2,4-dimethylhept-1-ene; decane or benzoic acid; methyl ester; or combinations thereof remains the same in isolated protein prepared from pulse flour that are heat treated when compared to the amounts of the compounds present in isolated protein prepared from pulse flour that has not been heat treated.

In one embodiment, the amount of 2,4-dimethylhept-1-ene; benzoic acid, methyl ester; or decane present in an isolated protein prepared from pulse treated with dry heat (without steam) does not decrease when compared to protein prepared from pulse flour made from unroasted pulses.

In one embodiment, the amount of 2,4-dimethylhept-1-ene; benzoic acid, methyl ester; or decane present in an isolated protein prepared from pulse treated with steam heat decreases when compared to isolate protein prepared from pulse flour made from unroasted pulses.

In one embodiment, a method of manufacturing a heat treated pulse flour is provided. In an embodiment, the method comprises incubating dehulled pulse or undehulled pulse at a desired temperature for a desired amount of time.

In one embodiment, the method comprises incubating undehulled pulse in a solvent at a desired temperature for a desired amount of time to remove the hulls. In this embodiment, the incubation of the pulse in the solvent removes the hull from the pulse.

In one embodiment, the heat treatment method comprises exposing the pulse, either dehulled or undehulled, in the absence of solvent, to one or more heating zones for a desired amount of time. The temperature of one heating zone may be different than the temperature of another heating zone. Optionally, after treatment in the one or more heating zones, the pulses are exposed to a cooling zone to cool the heat treated pulse to a desired temperature. The heat treated pulse is milled to prepare the heat treated pulse flour. The heat treated pulse flour comprises volatile small molecule compounds, wherein the amount of volatile small molecule compounds present in the heat treated pulse flour is increased or decreased as compared to the amount of small molecule compounds present in a pulse flour that is not heat treated.

In an embodiment, the pulse is exposed to steam in the one or more heating zones. The temperature of the steam is at a desired temperature of between 100° C. to 500° C.

In an embodiment, the temperature of the one or more heating zones is at a desired temperature. The desired temperature of the one or more heating zones in one embodiment is between 50° C. to 300° C.

In one embodiment, the temperature of a first heating zone is lower or higher than the temperature of a second heating zone. In an embodiment, the temperature of a first heating zone is between 110° C. to 150° C. In an embodiment, the temperature of a second heating zone is between 180° C. to 225° C.

In an embodiment, the temperature of the cooling zone is between 10° C. to 100° C.

In an embodiment, the amount of time that the pulse is exposed to heat (residence time) is determined by the skilled worker. In one embodiment, the residence time of the pulse in the one or more heating zones is between 1 and 60 minutes. In one embodiment, the residence time of the pulse in the one or more heating zones can be the same or different. In one embodiment, the residence time of the pulse in the first heating zone is shorter or longer than the residence time of the pulse in the second heating zone. In yet another embodiment, the residence time of the pulse in the cooling zone can be determined by the skilled worker. In an embodiment, the residence time of the pulse in the cooling zone is between 1 minute and 60 minutes.

In an embodiment, the method of manufacturing a heat treated pulse flour provided herein, small molecules present in the heat treated pulse flour decrease, increases or remains the same. In an embodiment, the amount of at least one small molecule compound is increased or decreased when compared to a pulse flour that has not been heat treated.

In one embodiment of the method of manufacturing heat treated pulse flour, the volatile small molecule compound present in the pulse flour is selected from the group consisting of heptane; 3-methyl, 1-butanol; 4-methyl heptane; 1-pentanol; 2,4-dimethylhept-1-ene; hexanal; benzoic acid, methyl ester; decane; 2-methyl; 1-pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; f3-ionone; undecanal; methyleugenol; 3-carene; dodecane; or combinations thereof.

In an embodiment of the method of manufacturing heat treated pulse flour, the amount of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; β-ionone; undecanal; methyleugenol; or combinations thereof present in the heat treated pulse flour is decreased as compared to the amount of small molecule compounds present in a pulse flour that is not heat treated.

In an embodiment of the method of manufacturing heat treated pulse flour, the amount of volatile small molecule compounds present in the heat treated pulse flour is increased as compared to the amount of small molecule compounds present in a pulse flour that is not heat treated.

In one embodiment of the method of manufacturing heat treated pulse flour, the amount of 3-carene or dodecane present in the heat treated pulse flour is increased as compared to the amount of 3-carene or dodecane present in a pulse flour that is not heat treated.

In one embodiment of the method of manufacturing heat treated pulse flour, the amount of 2,4-dimethylhept-1-ene; decane or benzoic acid, methyl ester present in the heat treated pulse flour remains the same as compared to the amount of 2,4-dimethylhept-1-ene; decane or benzoic acid, methyl ester present in a pulse flour that is not heat treated.

In one embodiment of the method of manufacturing heat treated pulse flour, the amount of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; β-ionone; undecanal; methyleugenol; or combinations thereof is decreased while concurrently the amount of 3-carene or dodecane is increased and/or concurrently the amount of 2,4-dimethylhept-1-ene; decane or benzoic acid, methyl ester remains the same compared to pulse flour that has not been heat treated.

In one embodiment of the method of manufacturing heat treated pulse flour, the amount of 2,4-dimethylhept-1-ene; benzoic acid, methyl ester; or decane present when the pulse is treated with dry heat (without steam) does not decrease when compared to pulse flour made from unroasted pulses.

In one embodiment of the method of manufacturing heat treated pulse flour, the amount of 2,4-dimethylhept-1-ene; benzoic acid, methyl ester; or decane present when the pulse is treated with steam heat decreases when compared to pulse flour made from unroasted pulses.

Other embodiments will become apparent from a review of the ensuing detailed description.

### BRIEF DESCRIPTION OF THE DRAWINGS

FIG. 1 depicts a process diagram for ultrafiltration purification of plant proteins.

FIG. 2A and FIG. 2B shows the decreases in the amounts of the identified VOCs in unroasted mung bean flour, heat treated mung bean flour, and heat treated and steamed mung bean flour.

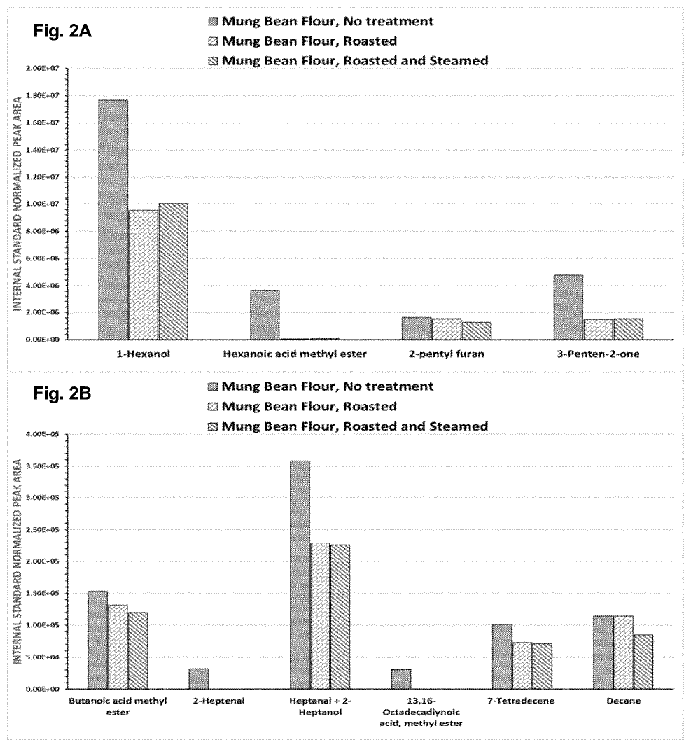

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Definitions

As used herein, the singular forms “a,” “an,” and “the” include the plural referents unless the context clearly indicates otherwise.

The term “reduce”, “reduced”, “depleted”, “decreased” or similar terms indicates a lessening or decrease of an indicated value relative to a reference value. In some embodiments, the term “reduce” (including “reduction”) refers to a lessening or a decrease of an indicated value to a reference value. When used in reference to volatile small molecule compound, “reduced” means that the amounts or concentrations of one or more small molecule compounds present in the heat treated pulse flour is decreased, reduced or lowered as compared to a pulse flour that has not been exposed to heat.

The term “increase”, “increased”, “enriched” or similar terms indicates an increase or increasing of an indicated value relative to a reference value. In some embodiments, the term “increase” (including “increasing”) refers to an increase of an indicated value. When used in reference to volatile small molecule compound, “increased” means that the amounts or concentrations of one or more small molecule compounds present in the heat treated pulse flour is higher as compared to a pulse flour that has not been exposed to heat.

As used herein, the term “eggs” includes but is not limited to chicken eggs, other bird eggs (such as quail eggs, duck eggs, ostrich eggs, turkey eggs, bantam eggs, goose eggs), and fish eggs such as fish roe. Typical food application comparison is made with respect to chicken eggs.

As used herein, “molecular weight,” “molecular size” or similar expressions refer to the molecular mass of compounds, such as proteins, expressed as dalton (Da) or kilodalton (kDa). The molecular weight of a compound can be precise or can be an average molecular mass. For example, the molecular weight of a discrete compound, such as NaCl or a specific protein can be precise. For the molecular sizes of protein isolates of the invention, an average molecular mass is typically used. For example, protein isolates obtained in the retentate fraction of a purification process using an ultrafiltration membrane having a molecular weight cut-off of 10 kDa are depleted in proteins (and other compounds) that have an average molecular weight of less 10 kDa. The retentate fraction from a 10 kDa UF membrane can also be described as being enriched in proteins (and other compounds) that have an average molecular weight of greater than 10 kDa. The permeate fraction of a purification process using an ultrafiltration membrane having a molecular weight cut-off of 10 kDa is enriched in proteins (and other compounds) that have an average molecular weight of less than 10 kDa. The permeate fraction from a 10 kDa UF membrane can also be described as being depleted in proteins (and other compounds) that have an average molecular weight of greater than 10 kDa.

As used herein, “plant source of the isolate” refers to a whole plant material such as whole mung bean or other pulse, or from an intermediate material made from the plant, for example, a dehulled bean, a undehulled bean, a flour, a powder, a meal, ground grains, a cake (such as, for example, a defatted or de-oiled cake), or any other intermediate material suitable to the processing techniques disclosed herein to produce a purified protein isolate.

The term “dehulled” or “hulled” when used to describe a pulse means a pulse in which the hull of the pulse has been removed.

The term “unhulled” when used to describe a pulse means a pulse in which the hull of the pulse has not been removed.

The term “transglutaminase” refers to an enzyme (R-glutamyl-peptide:amine glutamyl transferase) that catalyzes the acyl-transfer between γ-carboxyamide groups and various primary amines, classified as EC 2.3.2.13. It is used in the food industry to improve texture of some food products such as dairy, meat and cereal products. It can be isolated from a bacterial source, a fungus, a mold, a fish, a mammal and a plant.

The terms “majority” or “predominantly” with respect to a specified component, e.g., small molecule compound, refer to the component having at least 50% by weight of the referenced batch, process stream, food formulation or composition.

Unless indicated otherwise, percentage (%) of ingredients refer to total % by weight typically on a dry weight basis unless otherwise indicated.

The term “purified protein isolate”, “protein isolate”, “isolate”, “protein extract”, “isolated protein” or “isolated polypeptide” refers to a protein fraction, a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). One or more proteins or fractions may be partially removed or separated from residual source materials and/or non-solid protein materials and, therefore, are non-naturally occurring and are not normally found in nature. A polypeptide or protein may also be rendered substantially free of naturally associated components by isolation, using protein purification techniques known in the art and as described herein. A polypeptide that is chemically synthesized or synthesized in a cellular system different from the cell from which it naturally originates will be “isolated” from its naturally associated components. As thus defined, “isolated” does not necessarily require that the protein, polypeptide, peptide or oligopeptide so described has been physically removed from its native environment.

The term “heat treated pulse flour” or “heat treated flour” refers to milled pulses that have been exposed to heat. The milling can occur before or after heat treatment. The term also refers to milled pulses that have been exposed to steam before, during or after exposure of the pulses to heat.

The term “mill, “milling,” “milled” and the like refers to the process of or the product produced by reducing the size of a pulse by grinding, crushing, macerating or other methods.

The terms “volatile small molecule compound” or “small molecule compound” refers to compounds present in the pulse before, during or after heat treatment of the pulse.

The term “heating zone,” “heating portion,” heating section,” and the like refers to one or more zones of a dryer in which the temperature of a heating zone can be independently controlled from the temperature of other heating zones.

The term “cooling zone,” “cooling portion,” cooling section,” and the like refers to one or more zones of a dryer in which the temperature of a cooling zone can be independently controlled from the temperature of other cooling zones.

As used herein the term “residence time” refers to the amount of time that a pulse resides in the one or more heating zones or the one or more cooling zones.

As used herein “volatile small molecule compound(s)” or “small molecule compound” refers to compound having a molar mass or molecular weight of less than 2,000 Da, less than 1500 Da, less that 1,000 Da, less than 750 Da or less than 500 Da.

As used herein “pulse” refers to legumes that are grown and harvested for their dry seed and grown as food.

### Heat Treated Pulse Flour

The present disclosure provides heat treated pulse flour.

Disclosed herein are heat treated pulse flours wherein the volatile small molecule compounds present in the heat treated pulse flour is decreased, increased or is unaltered as compared to the amount of small molecule compounds present in a pulse flour that is not heat treated. The presence and concentrations of the one or more volatile small molecule compounds alters the flavor and/or odor of the flour. The changes in the amounts of the volatile small molecule compounds in the heat treated flour alters the flavor and/or odor of the proteins isolated from the heat treated pulse flour. The flavor and/or odor of a food product, for example an egg substitute, that comprises protein isolates obtained from the heat treated pulse protein is thereby improved.

In an embodiment, the amount of at least one, two, three, four, five, six, seven, eight, nine ten, or more than ten volatile small molecule compound present in the heat treated pulse is decreased or increased as compared to a non-heat treated pulse flour.

In an embodiment, the amount or concentration of one or more volatile small molecule compounds present in the heat treated pulse flour is decreased as compared to the small molecule compounds present in a pulse flour that is obtained from a non-heat treated pulse.

In an embodiment, the amount or concentration of one or more volatile small molecule compounds present in the heat treated pulse flour is increased as compared to the small molecule compounds present in a pulse flour that obtained from a non-heat treated pulse.

In an embodiment, the amount or concentration of one or more volatile small molecule compounds present in the heat treated pulse flour is unaltered or remains the same as compared to the small molecule compounds present in a pulse flour that is obtained from a non-heat treated pulse.

In one embodiment, the amount or concentration of the one or more volatile small molecule compounds present in the heat treated pulse flour is lower as compared to a pulse flour that is obtained from a non-heat treated pulse, the amount of one or more volatile small molecule compounds present in the heat treated pulse flour is higher as compared to a pulse flour that is obtained from a non-heat treated pulse, and the amount of one or more small molecule compounds present in the heat treated pulse flour and the non-heat treated flour is not altered. In this embodiment, the identities of the one or more volatile small molecule compounds that are increased is different than the identities of the one or more volatile small molecule compounds that are decreased or not altered.

In an embodiment, the volatile small molecule compound present in the heat treated flour is selected from the group consisting of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; 0-ionone; undecanal; methyleugenol; 3-carene; dodecane; or combinations thereof. The presence and concentrations of the one or more volatile small molecule compounds alters the flavor and/or odor of the heat treated flour. When protein are isolated from the isolated from the heat treated pulse flour, the flavor and/or odor of the pulse protein isolate is altered by the increases and/or decreases of the one or more volatile small molecule compounds. The flavor and/or odor of a food product, for example an egg substitute, that comprises protein isolates obtained from the heat treated pulse protein is thereby improved.

In an embodiment, the amount of the one or more volatile small molecule compounds in the heat treated flour is decreased by 1-10000 fold (1 X-10,000 X), between 1 X-5,000 X, between 1 X-4,000 X, between 1 X-3,000 X, between 1 X-2,000 X, between 1 X-1,000 X, between 1 X-500 X, between 1 X-400 X, between 1 X-300 X, between 1 X-200 X, between 1 X-100 X, between 1 X-75 X, between 1 X-50 X, between 1 X-30 X, between 1 X-20 X, between 1 X-10 X, between 1 X-5 X, between 1 X-3 X, or between 1 X-2 X as compared to a non-heat treated flour. In an embodiment the amount of the one or more volatile small molecule compounds in the heat treated flour is decreased by between: 1% and 5%, 5% and 10%, 10% and 15%, 15% and 20%, 20% and 25%, 25% and 30%, 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, 95% and 99%, 5% and 25%, 25% and 50%, 50% and 75%, or 75% and 95%.

In an embodiment, the amount of the one or more volatile small molecule compounds in the heat treated flour is increased by 1-10000 fold (1 X-10,000 X), between 1 X-5,000 X, between 1 X-4,000 X, between 1 X-3,000 X, between 1 X-2,000 X, between 1 X-1,000 X, between 1 X-500 X, between 1 X-400 X, between 1 X-300 X, between 1 X-200 X, between 1 X-100 X, between 1 X-75 X, between 1 X-50 X, between 1 X-30 X, between 1 X-20 X, between 1 X-10 X, between 1 X-5 X, between 1 X-3 X, or between 1 X-2 X as compared to a non-heat treated flour. In an embodiment the amount of the one or more volatile small molecule compounds in the heat treated flour is decreased by between: 1% and 5%, 5% and 10%, 10% and 15%, 15% and 20%, 20% and 25%, 25% and 30%, 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, 95% and 99%, 5% and 25%, 25% and 50%, 50% and 75%, or 75% and 95%.

In In an embodiment, the amount of the one or more volatile small molecule compounds in the heat treated flour is unaltered.

In an embodiment, flavor and/or odor properties of the volatile small molecules are as described herein.

In one embodiment, the heat treated pulse flour is made from a pulse selected from the group consisting of dry beans, lentils, mung beans, fava beans, dry peas, chickpeas, cowpeas, bambara beans, pigeon peas, lupins, vetches, adzuki, common beans, fenugreek, long beans, lima beans, runner beans, tepary beans, soy beans, and mucuna beans. In an embodiment the heat treated pulse flour is of the genus Vigna. In another embodiment, the heat treated pulse flour is of the species Vigna radiata or Vigna radiata.

In an embodiment, the pulse is not dehulled (unhulled), that is, a pulse in which the hull has not been removed from the pulse.

In an embodiment, the pulse is dehulled, that is, a pulse in which the hull has been removed from the pulse. The pulse is dehulled by contacting the pulse with a solvent for a desired amount of time. In one embodiment, the solvent used for dehulling the pulse is water, ethanol, oil, or other solvents. Optionally, salts such as sodium salts, potassium salts, ammonium salts or other salts can be added to the solvent. Without being bound by theory, it is believed that the incubation of the undehulled pulse removes the hull and also removes volatile small molecule compounds from the pulse.

In one embodiment, the temperature of the solvent for dehulling is maintained at a temperature of between: 20° C. to 100° C., 20° C. to 95° C., 20° C. to 90° C., 20° C. to 85° C., 20° C. to 80° C., 20° C. to 75° C., 20° C. to 70° C., 20° C. to 65° C., 20° C. to 60° C., 20° C. to 55° C., 20° C. to 50° C., 20° C. to 45° C., 20° C. to 40° C., 20° C. to 35° C., 20° C. to 30° C., 20° C. to 25° C., 25° C. to 100° C., 25° C. to 95° C., 25° C. to 90° C., 25° C. to 85° C., 25° C. to 25° C., 25° C. to 75° C., 25° C. to 70° C., 25° C. to 65° C., 25° C. to 60° C., 25° C. to 55° C., 25° C. to 50° C., 25° C. to 45° C., 25° C. to 40° C., 25° C. to 35° C., 25° C. to 30° C., 30° C. to 40° C., 40° C. to 50° C., 50° C. to 60° C., 60° C. to 70° C., 70° C. to 80° C., 80° C. to 90° C., or 90° C. to 100° C.

In one embodiment, the time that the pulse is contacted with the solvent to remove the hull is between: 30 minutes to 24 hours, 30 minutes to 24 hours, 30 minutes to 20 hours, 30 minutes to 15 hours, 30 minutes to 10 hours, 30 minutes to 9 hours, 30 minutes to 8 hours, 30 minutes to 7 hours, 30 minutes to 6 hours, 30 minutes to 5 hours, 30 minutes to 4 hours, 30 minutes to 3 hours, 30 minutes to 2 hours, 30 minutes to 1 hour, 1 hour to 20 hours, 1 hour to 15 hours, 1 hour to 10 hours, 1 hour to 9 hours, 1 hour to 8 hours, 1 hour to 7 hours, 1 hour to 6 hours, 1 hour to 5 hours, 1 hour to 4 hours, 1 hour to 3 hours, 1 hour to 2 hours, 2 hours to 24 hours, 2 hours to 24 hours, 2 hours to 20 hours, 2 hours to 15 hours, 2 hours to 10 hours, 2 hours to 9 hours, 2 hours to 8 hours, 2 hours to 7 hours, 2 hours to 6 hours, 2 hours to 5 hours, 2 hours to 4 hours, or 2 hours to 3 hours.

In an embodiment, the presence and/or concentrations of the volatile small molecule compounds are isolated and detected by methods known to the skilled artisan. Isolation of the volatile small molecule compounds can be achieved by headspace gas analysis, in-tube extraction dynamic headspace (ITEX-DHS), stirbar sorptive extraction (SBSE), or solid phase microextraction (SPME). Once the compounds are obtained they are analyzed by gas chromatography (GC), liquid chromatography (LC), high performance liquid chromatography (HPLC), mass spectroscopy (MS), nuclear magnetic resonance (NMR), infrared spectroscopy (IR), optical spectroscopy, and other techniques such as GC/MS.

Isolated Pulse Proteins Obtained from Heat Treated Pulse Flours

In one embodiment, provided are isolated proteins obtained from heat treated pulse flour.

The protein isolates disclosed herein are obtained from heat treated pulse flours wherein the volatile small molecule compounds present in the protein isolate is decreased, increased or is unaltered as compared to the amount of small molecule compounds present in a protein isolate obtained pulse flour that is not heat treated. The presence and concentrations of the one or more volatile small molecule compounds alters the flavor and/or odor of the protein isolate. The flavor and/or odor of a food product, for example an egg substitute, that comprises protein isolates obtained from the heat treated pulse protein is thereby improved.

In an embodiment, the amount or concentration of one or more volatile small molecule compounds present in the protein isolate is decreased as compared to the small molecule compounds present in a protein isolate that is obtained from a non-heat treated pulse.

In an embodiment, the amount or concentration of one or more volatile small molecule compounds present in the protein isolate is increased as compared to the small molecule compounds present in a protein isolate that obtained from a non-heat treated pulse.

In an embodiment, the amount or concentration of one or more volatile small molecule compounds present in the protein isolate is unaltered or remains the same as compared to the small molecule compounds present in protein isolate that is obtained from a non-heat treated pulse.

In one embodiment, the amount or concentration of the one or more volatile small molecule compounds present in the protein isolate is lower as compared to a protein isolate that is obtained from a non-heat treated pulse, the amount of one or more volatile small molecule compounds present in the protein isolate is higher as compared to a protein isolate that is obtained from a non-heat treated pulse, and the amount of one or more small molecule compounds present in the protein isolate obtained from the heat treated flour and the protein isolate obtained from the non-heat treated flour is not altered. In this embodiment, the identities of the one or more volatile small molecule compounds that are increased is different than the identities of the one or more volatile small molecule compounds that are decreased or not altered.

In an embodiment, the volatile small molecule compound present in the protein isolate is selected from the group consisting of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; β-ionone; undecanal; methyleugenol; 3-carene; dodecane; and combinations thereof. The presence and concentrations of the one or more volatile small molecule compounds alters the flavor and/or odor of the heat treated flour. The flavor and/or odor of a food product, for example an egg substitute, that comprises protein isolates obtained from the heat treated pulse protein is thereby improved.

In an embodiment the amount of the one or more volatile small molecule compounds in the protein isolate is decreased by 1-10000 fold (1 X-10,000 X), between 1 X-5,000 X, between 1 X-4,000 X, between 1 X-3,000 X, between 1 X-2,000 X, between 1 X-1,000 X, between 1 X-500 X, between 1 X-400 X, between 1 X-300 X, between 1 X-200 X, between 1 X-100 X, between 1 X-75 X, between 1 X-50 X, between 1 X-30 X, between 1 X-20 X, between 1 X-10 X, between 1 X-5 X, between 1 X-3 X, or between 1 X-2 X as compared to a non-heat treated flour. In an embodiment the amount of the one or more volatile small molecule compounds in the heat treated flour is decreased by between: 1% and 5%, 5% and 10%, 10% and 15%, 15% and 20%, 20% and 25%, 25% and 30%, 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, 95% and 99%, 5% and 25%, 25% and 50%, 50% and 75%, or 75% and 95%.

In an embodiment the amount of the one or more volatile small molecule compounds in the protein isolate is increased by 1-10000 fold (1 X-10,000 X), between 1 X-5,000 X, between 1 X-4,000 X, between 1 X-3,000 X, between 1 X-2,000 X, between 1 X-1,000 X, between 1 X-500 X, between 1 X-400 X, between 1 X-300 X, between 1 X-200 X, between 1 X-100 X, between 1 X-75 X, between 1 X-50 X, between 1 X-30 X, between 1 X-20 X, between 1 X-10 X, between 1 X-5 X, between 1 X-3 X, or between 1 X-2 X as compared to a non-heat treated flour. In an embodiment the amount of the one or more volatile small molecule compounds in the heat treated flour is increased by between: 1% and 5%, 5% and 10%, 10% and 15%, 15% and 20%, 20% and 25%, 25% and 30%, 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, 95% and 99%, 5% and 25%, 25% and 50%, 50% and 75%, or 75% and 95%.

In an embodiment the amount of the one or more volatile small molecule compounds in the protein isolate is unaltered or remains the same.

In an embodiment, various flavor and/or odor properties of the volatile small molecules have been identified. The disclosures provided herein provide the flavor and/or odor properties of the volatile small molecules.

In one embodiment, the protein isolate is obtained from a pulse selected from the group consisting of dry beans, lentils, mung beans, fava beans, dry peas, chickpeas, cowpeas, bambara beans, pigeon peas, lupins, vetches, adzuki, common beans, fenugreek, long beans, lima beans, runner beans, tepary beans, soy beans, and mucuna beans. In an embodiment the protein isolate is obtained from the genus Vigna. In another embodiment, the protein isolate is obtained from the species Vigna radiata or Vigna radiata.

In an embodiment, the presence and/or concentrations of the volatile small molecule compounds are isolated and detected by methods known to the skilled artisan. Isolation of the volatile small molecule compounds can be achieved by headspace gas analysis, in-tube extraction dynamic headspace (ITEX-DHS), stirbar sorptive extraction (SBSE), or solid phase microextraction (SPME). Once the compounds are obtained they are analyzed by gas chromatography (GC), liquid chromatography (LC), high performance liquid chromatography (HPLC), mass spectroscopy (MS), nuclear magnetic resonance (NMR), infrared spectroscopy (IR), optical spectroscopy, and other techniques such as GC/MS.

### Methods of Producing Heat Treated Pulse Flours

The present disclosure provides method of producing heat treated pulse flours. The pulse flour is prepared, in one embodiment, by exposing pulses to heat and milling the heat treated pulse. In an embodiment, the pulse is exposed to steam before, during or after exposure to heat and milled to prepare the heat treated pulse flour. In one embodiment, the pulse is exposed to dry heat, that is, the pulse is exposed to heat without the use of steam. In one embodiment, the pulse is exposed to heat with the use of steam. The exposure of the pulse to heat by use of steam is referred to as “steam stripping.” When the term “heat” is used herein, the term refers to dry heat, steam heat or both.

In another embodiment, the heat treated pulse flour is prepared by first milling pulses at ambient temperatures and next exposing the milled pulse to heat to prepare a heat treated pulse flour.

In an embodiment the heat treated pulse flour prepared by the methods disclosed herein comprises volatile small molecule compounds, wherein the amount or concentrations of volatile small molecule compounds present in the heat treated pulse flour is decreased, increased or is not altered as compared to the amount of small molecule compounds present in a pulse flour that is not heat treated. The identities and changes to the amount or concentrations of the volatile small molecules present in the heat treated pulses are described elsewhere in this application.

In an embodiment, the methods provided herein produces heat treated pulse flour in which the amount of at least one, two, three, four, five, six, seven, eight, nine ten, or more than ten volatile small molecule compound present in the heat treated pulse is decreased or increased as compared to a non-heat treated pulse flour.

In an embodiment, the heat treatment of the pulses is accomplished by exposing the pulses to one or more heating zones in a dryer. There can be one, two, three, four or more heating zones in the dryer. The temperatures of the one or more heating zones can be individually controlled. The temperature of one or a first heating zone may be different than the temperature of another or second zone. In one embodiment, the temperature of the first heating zone is lower than the temperature of another heating zone, for example, the second heating zone or a third heating zone. In one embodiment, the temperature of the first heating zone is higher than the temperature of another heating zone, for example, the second heating zone or a third heating zone. The skilled worker will understand that each heating zone can be controlled individually and that the temperature of each heating zone can be higher or lower than another heating zone.

In one embodiment, the temperature of the one or more heating zones is individually between: 75° C. to 500° C., 100° C. to 500° C., 100° C. to 475° C., 100° C. to 450° C., 100° C. to 425° C., 100° C. to 400° C., 100° C. to 375° C., 100° C. to 350° C., 100° C. to 325° C., 100° C. to 300° C., 100° C. to 275° C., 100° C. to 250° C., 100° C. to 225° C., 100° C. to 200° C., 100° C. to 175° C., 100° C. to 150° C., 100° C. to 125° C., 125° C. to 400° C., 125° C. to 375° C., 125° C. to 350° C., 125° C. to 300° C., 125° C. to 275° C., 125° C. to 250° C., 125° C. to 250° C., 125° C. to 225° C., 125° C. to 200° C., 125° C. to 175° C., 125° C. to 150° C., 150° C. to 400° C., 150° C. to 350° C., 150° C. to 250° C., or 150° C. to 200° C.

In an embodiment, the temperature of steam is between: 100° C. to 500° C., 100° C. to 400° C., between 100° C. to 300° C., 100° C. to 200° C., 100° C. to 150° C., 150° C. to 500° C., 150° C. to 400° C., 150° C. to 350° C., 150° C. to 300° C., 150° C. to 250° C., 150° C. to 200° C., 200° C. to 500° C., 200° C. to 400° C., 200° C. to 350° C., 200° C. to 300° C., 200° C. to 250° C., 250° C. to 500° C., 250° C. to 400° C., 250° C. to 350° C., 250° C. to 300° C., 300° C. to 500° C., 300° C. to 400° C., 300° C. to 350° C., or 350° C. to 400° C.

In one embodiment, the amount of steam that is applied during the heat treatment process is between: 0.5% to 20% by weight of the beans. For example, if 100 kg of beans are heat treated, between 0.5 kg and 20 kg of steam is added before, during or after heat treatment. In an embodiment, the amount of steam by weight of beans is between: 0.5% to 20%, 0.5% to 18%, 0.5% to 15%, 0.5% to 13%, 0.5% to 10%, 0.5% to 9%, 0.5% to 8%, 0.5% to 7%, 0.5% to 6%, 0.5% to 5%, 0.5% to 4%, 0.5% to 3%, 0.5% to 2%, 0.5% to 1%, 1% to 18%, 1% to 15%, 1% to 13%, 1% to 10%, 1% to 9%, 1% to 8%, 1% to 7%, 1% to 6%, 1% to 5%, 1% to 4%, 1% to 3%, 1% to 2%, 2% to 18%, 2% to 15%, 2% to 13%, 2% to 10%, 2% to 9%, 2% to 8%, 2% to 7%, 2% to 6%, 2% to 5%, 2% to 4%, 2% to 3%, 5% to 18%, 5% to 15%, 5% to 13%, 5% to 10%, 5% to 9%, 5% to 8%, 5% to 7%, or 5% to 6%.

In one embodiment, the time that the pulses are exposed to heat and/or steam in the one or more heating zones, also known as residence time, is between: 5 seconds and 30 minutes 1 minute and 25 minutes, between 1 minute and 20 minutes, between 1 minute and 15 minutes, between 1 minute and 10 minutes, between 1 minute and 8 minutes, between 1 minute and 7 minutes, between 1 minute and 6 minutes, between 1 minute and 5 minutes, between 1 minute and 4 minutes, between 1 minute and 3 minutes, between 1 minute and 2 minutes, between 2 minutes and 30 minutes, between 2 minutes and 20 minutes, between 2 minutes and 20 minutes, between 2 minutes and 5 minutes, between 3 minutes and 30 minutes, between 3 minutes and 20 minutes, between 3 minutes and 10 minutes, between 3 minutes and 5 minutes, between 5 minutes and 30 minutes, between 5 minutes and 30 minutes, between 5 minutes and 30 minutes, between 5 minutes and 30 minutes, between 5 minutes and 20 minutes, or between 5 minutes and 10 minutes.

In one embodiment, the temperature of the one or more cooling zones is individually at ambient temperature, between: 10° C. to 75° C., 10° C. to 70° C., 10° C. to 60° C., 10° C. to 50° C., 10° C. to 40° C., 10° C. to 30° C., 10° C. to 20° C., 20° C. to 100° C., 20° C. to 75° C., 20° C. to 50° C., 20° C. to 40° C., or 20° C. to 30° C., 30° C. to 70° C., 30° C. to 60° C., 30° C. to 50° C., 30° C. to 40° C.

In one embodiment, the time that the pulses are exposed to the one or more cooling zones, also known as residence time, is between: 5 seconds and 60 minutes, 5 seconds and 50 minutes, 5 seconds and 40 minutes, 5 seconds and 30 minutes, 5 seconds and 25 minutes, 5 seconds and 20 minute, 5 seconds and 15 minutes, 5 seconds and 10 minutes, 5 seconds and 5 minutes, 5 seconds and 3 minutes, 5 seconds and 2 minutes, 5 seconds and 1 minute, 10 seconds and 60 minutes, 10 seconds and 50 minutes, 10 seconds and 40 minutes, 10 seconds and 30 minutes, 10 seconds and 25 minutes, 10 seconds and 20 minute, 10 seconds and 15 minutes, 10 seconds and 10 minutes, 10 seconds and 5 minutes, 10 seconds and 3 minutes, 10 seconds and 2 minutes, 10 seconds and 1 minute, 30 seconds and 60 minutes, 30 seconds and 50 minutes, 30 seconds and 40 minutes, 30 seconds and 30 minutes, 30 seconds and 25 minutes, 30 seconds and 20 minute, 30 seconds and 15 minutes, 30 seconds and 10 minutes, 30 seconds and 5 minutes, 30 seconds and 3 minutes, 30 seconds and 2 minutes, 30 seconds and 1 minute, 40 seconds and 60 minutes, 40 seconds and 50 minutes, 40 seconds and 40 minutes, 40 seconds and 30 minutes, 40 seconds and 25 minutes, 40 seconds and 20 minute, 40 seconds and 15 minutes, 40 seconds and 10 minutes, 40 seconds and 5 minutes, 40 seconds and 3 minutes, 40 seconds and 2 minutes, 40 seconds and 1 minute, 50 seconds and 60 minutes, 50 seconds and 50 minutes, 50 seconds and 40 minutes, 50 seconds and 30 minutes, 50 seconds and 25 minutes, 50 seconds and 20 minute, 50 seconds and 15 minutes, 50 seconds and 10 minutes, 50 seconds and 5 minutes, 50 seconds and 3 minutes, 50 seconds and 2 minutes, 50 seconds and 1 minute, 1 minute and 60 minutes, 1 minute and 50 minutes, 1 minute and 40 minutes, 1 minute and 30 minutes, 1 minute and 20 minutes, 1 minute and 10 minutes, 1 minute and 5 minutes, 2 minutes and 60 minutes, 2 minutes and 50 minutes, 2 minutes and 40 minutes, 2 minutes and 30 minutes, 2 minutes and 20 minutes, 2 minutes and 10 minutes, 2 minutes and 5 minutes, 3 minutes and 30 minutes, 3 minutes and 20 minutes, 3 minutes and 20 minutes, 3 minutes and 10 minutes, 3 minutes and 5 minutes or 3 minutes and 4 minutes.

In one embodiment, the methods disclosed are used to prepare heat treated pulse flour. The pulse is selected from the group consisting of dry beans, lentils, mung beans, fava beans, dry peas, chickpeas, cowpeas, bambara beans, pigeon peas, lupins, vetches, adzuki, common beans, fenugreek, long beans, lima beans, runner beans, tepary beans, soy beans, and mucuna beans. In an embodiment the heat treated pulse flour is of the genus Vigna. In another embodiment, the heat treated pulse flour is of the species Vigna radiata or Vigna radiata.

### Methods of Producing Pulse Protein Isolates

The present disclosure includes methods of preparing pulse protein isolates (e.g., mung bean protein isolates) using ultrafiltration techniques or by isoelectric precipitation. The pulse protein isolates prepared by these methods have characteristics that are advantageous for the preparation of food product compositions, as discussed in greater detail below.

An exemplary embodiment of a method for producing pulse protein isolates is through ultrafiltration. In one embodiment of UF protein isolation, a heat treated pulse is milled into flour. (The milled heat treated pulse flour is then subjected to protein extraction by producing a flour slurry in an aqueous solution. Starch solids are separated from the flour slurry to produce a protein-rich fraction. The protein-rich fraction is then introduced into an ultrafiltration process) to produce a purified protein

In some embodiments, the methods for producing the pulse protein isolate comprise (a) extracting protein from a milled composition comprising pulse proteins in an aqueous solution at a pH of from about 1 to about 9 to produce a protein rich fraction containing extracted pulse proteins, (b) applying the protein rich fraction to an ultrafiltration process comprising a semi-permeable membrane to separate a retentate fraction from a permeate fraction based on molecular size at a temperature of from about 5° C. to about 60° C., (c) collecting the retentate fraction containing the pulse protein isolate. In various embodiments, the methods may further comprise: dehulling and milling pulses to produce the milled composition comprising pulse proteins; drying the pulses prior to milling; adjusting the pH and/or conductivity of the retentate fraction; heating the retentate fraction to pasteurize the pulse proteins; and/or removing water or drying the retentate fraction and/or the pulse protein isolate.

In various embodiments, the pulse proteins may be isolated from any pulse, including dry beans, lentils, faba beans, dry peas, chickpeas, cowpeas, bambara beans, pigeon peas, lupins, vetches, adzuki, common beans, fenugreek, long beans, lima beans, runner beans, tepary beans, soy beans, or mucuna beans. In various embodiments, the pulse proteins may be isolated from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius. In some embodiments, the pulse proteins are isolated from mung beans (Vigna radiata). In other embodiments, the milled composition may comprise almonds and other nuts, seeds such as sesame seeds, sunflower seeds, and other commonly consumed nuts, fruits and seeds.

In various embodiments, the methods discussed above or herein produce a pulse protein isolate comprising pulse proteins having a molecular size of less than 100 kilodaltons (kDa). In some embodiments, the methods produce a pulse protein isolate comprising pulse proteins having a molecular size of less than 95 kDa, 90 kDa, 85 kDa, 80 kDa, 75 kDa, 70 kDa, 65 kDa, 60, kDa, 55 kDa, 50 kDa, 45 kDa, 40 kDa, 35 kDa, 30 kDa, 25 kDa, 20 kDa or 15 kDa. In various embodiments, the methods produce a pulse protein isolate comprising pulse proteins having a molecular size of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa. Unless otherwise noted, references to a pulse protein isolate comprising pulse proteins having a specified molecular weight does not exclude the possibility that the same pulse protein isolate also contains pulse proteins of other molecular weights.

In various embodiments, the methods discussed above or herein produce a pulse protein isolate comprising pulse proteins enriched in proteins having a molecular size of greater than 5 kilodaltons (kDa). In some embodiments, the methods produce a pulse protein isolate comprising pulse proteins enriched in proteins having a molecular size of greater than 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa or 95 kDa Unless otherwise noted, references to a pulse protein isolate comprising pulse proteins having a specified molecular weight does not exclude the possibility that the same pulse protein isolate or retentate fraction also contains pulse proteins of other molecular weights.

In various embodiments, the methods discussed above or herein produce a pulse protein isolate having a storage modulus of from 25 Pa to 500 Pa at a temperature between 90° C. and 95° C., as measured by dynamic oscillatory rheology using a rheometer equipped with a flat parallel plate geometry of 40 mm in which the measured pulse protein isolate comprises 12% w/w protein and the storage modulus is recorded under 0.1% strain conditions at a constant angular frequency of 10 rad/s. In various embodiments, the methods discussed above or herein produce a pulse protein isolate having a storage modulus of less than 50 Pa at a temperature between 90° C. and 95° C., as measured by dynamic oscillatory rheology using a rheometer equipped with a flat parallel plate geometry of 40 mm in which the measured pulse protein isolate comprises 12% w/w protein and the storage modulus is recorded under 0.1% strain conditions at a constant angular frequency of 10 rad/s.

In various embodiments, the methods discussed above or herein produce a pulse protein isolate having a linear viscoelastic region of from 25 Pa to 1500 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C. In various embodiments, the methods discussed above or herein produce a pulse protein isolate having a linear viscoelastic region of less than 1000 Pa at up to 10% strain, as measured by dynamic oscillatory rheology using a rheometer equipped with a flat parallel plate geometry of 40 mm in which the measured pulse protein isolate comprises 12% w/w protein and the strain is carried out at a constant frequency of 10 rad/s at 50° C. In some embodiments, the methods produce a pulse protein isolate having a linear viscoelastic region of less than 500 Pa at up to 10% strain, or a linear viscoelastic region of less than 200 Pa at up to 10% strain.

### Dehulling, Drying and Milling

The pulse protein isolates (e.g., mung bean isolates) provided herein may be prepared from any suitable source of pulse protein, where the starting material is whole plant material (e.g., whole mung bean). In some cases, the methods may include dehulling the raw source material. In some such embodiments, raw pulse protein materials (e.g., mung beans) may be dehulled in one or more steps of pitting, soaking, and drying to remove the seed coat (husk) and pericarp (bran).

In an embodiment, heat treated pulses are prepared from pulses that are not dehulled.

In an embodiment, heat treated pulses are prepared from pulses that are dehulled. The pulse is dehulled by contacting the pulse with a solvent for a desired amount of time. In one embodiment, the solvent used for dehulling the pulse is water, ethanol, oil, or other solvents. Optionally, salts such as sodium salts, potassium salts, ammonium salts or other salts can be added to the solvent. Without being bound by theory, it is believed that the incubation of the undehulled pulse removes the hull and also removes volatile small molecule compounds from the pulse.

In one embodiment, the temperature of the solvent for dehulling is maintained at a temperature of between: 20° C. to 100° C., 20° C. to 95° C., 20° C. to 90° C., 20° C. to 85° C., 20° C. to 80° C., 20° C. to 75° C., 20° C. to 70° C., 20° C. to 65° C., 20° C. to 60° C., 20° C. to 55° C., 20° C. to 50° C., 20° C. to 45° C., 20° C. to 40° C., 20° C. to 35° C., 20° C. to 30° C., 20° C. to 25° C., 25° C. to 100° C., 25° C. to 95° C., 25° C. to 90° C., 25° C. to 85° C., 25° C. to 25° C., 25° C. to 75° C., 25° C. to 70° C., 25° C. to 65° C., 25° C. to 60° C., 25° C. to 55° C., 25° C. to 50° C., 25° C. to 45° C., 25° C. to 40° C., 25° C. to 35° C., 25° C. to 30° C., 30° C. to 40° C., 40° C. to 50° C., 50° C. to 60° C., 60° C. to 70° C., 70° C. to 80° C., 80° C. to 90° C., or 90° C. to 100° C.

In one embodiment, the time that the pulse is contacted with the solvent is between: 30 minutes to 24 hours, 30 minutes to 24 hours, 30 minutes to 20 hours, 30 minutes to 15 hours, 30 minutes to 10 hours, 30 minutes to 9 hours, 30 minutes to 8 hours, 30 minutes to 7 hours, 30 minutes to 6 hours, 30 minutes to 5 hours, 30 minutes to 4 hours, 30 minutes to 3 hours, 30 minutes to 2 hours, 30 minutes to 1 hour, 1 hour to 20 hours, 1 hour to 15 hours, 1 hour to 10 hours, 1 hour to 9 hours, 1 hour to 8 hours, 1 hour to 7 hours, 1 hour to 6 hours, 1 hour to 5 hours, 1 hour to 4 hours, 1 hour to 3 hours, 1 hour to 2 hours, 2 hours to 24 hours, 2 hours to 24 hours, 2 hours to 20 hours, 2 hours to 15 hours, 2 hours to 10 hours, 2 hours to 9 hours, 2 hours to 8 hours, 2 hours to 7 hours, 2 hours to 6 hours, 2 hours to 5 hours, 2 hours to 4 hours, or 2 hours to 3 hours.

The de-hulled material (e.g., mung beans in which the hulls have been removed) are then milled to produce a composition (e.g., flour) with a desired particle size distribution. The types of mills employed may include one or a combination of a hammer, pin, knife, burr, and air classifying mills.

Air classification is an industrial process in which materials are separated by a combination of density, size and/or shape. Dried materials such as pulse flours, for example mung bean flour, are introduced into an air classifier (air elutriator) where the flour particles are subjected to a column of rising air. The less dense flour particles are carried further in the air stream and separation of flour particles by density is achieved. The applicant has discovered that less dense pulse flour particles contain higher amounts of protein than the flour particles with higher density.

### Protein Extraction

The methods for producing the pulse protein isolate comprise extracting protein from a milled composition comprising pulse proteins in an aqueous solution at a pH of from about 1 to about 9 to produce a protein rich fraction containing extracted pulse proteins. In some embodiments, the aqueous solution has a pH of from about 4 to about 9. In some embodiments, the aqueous solution has a pH of from about 6 to about 10. In some embodiments, the aqueous solution has a pH of about 7 to about 9. In some embodiment, the aqueous solution has a pH of about 8. In various embodiments, the pH of the aqueous solution is about 1, 1.5, 2, 2.5, 3, 3.5, 4, 4.5, 5, 5.5, 6, 6.5, 7, 7.5, 8, 8.5, 9, 9.5 or 10. In some embodiments, the extraction is performed at a pH of 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or 9.5. The pH of the slurry may be adjusted with, e.g., a food-grade 50% sodium hydroxide solution to reach the desired extraction pH.

In some embodiments of the extraction step, an intermediate starting material, for example, a milled composition comprising pulse proteins (e.g., mung bean flour), is mixed with an aqueous solution to form a slurry. In some embodiments, the aqueous solution is water, for example soft water. The aqueous extraction may include creating an aqueous solution comprising one part of the source of the plant protein (e.g., flour) to about, for example, 3 to 15 parts aqueous extraction solution. Additional useful solid:liquid ratios for extraction include 1:2, 1:3, 1:4, 1:5, 1:6, 1:7, 1:8, 1:9, 1:10, 1:11, 1:12, 1:13, 1:14, 1:15. In some embodiments, extraction is performed using a solid:liquid ratio of 1:6.

In some cases, the aqueous solution comprises a salt. In some cases, the salt concentration is at least 0.01% w/v. In some cases, the salt concentration is at least 0.1% w/v. In some cases, the salt concentration is from 0.01% w/v to 5% w/v. In various embodiments, the salt concentration is 0.001%, 0.0025%, 0.005%, 0.0075%, 0.01%, 0.025%, 0.05%, 0.075%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0%. In various embodiments, the salt is selected from sodium chloride, sodium sulfate, sodium phosphate, ammonium sulfate, ammonium phosphate, ammonium chloride, potassium chloride, potassium sulfate, or potassium phosphate. In some embodiments, the salt is NaCl. In some embodiments, the aqueous solution does not comprise a salt.

In some cases, the aqueous extraction is performed at a desired temperature, for example, about 2-10° C. in a chilled mix tank to form the slurry. In some embodiments, the mixing is performed under moderate to high shear. In some embodiments, a food-grade de-foaming agent (e.g., KFO 402 Polyglycol) is added to the slurry to reduce foaming during the mixing process. De-foamers include, but are not limited to, polyglycol based de-foamers, vegetable oil based de-foamers, and silicone. In other embodiments, a de-foaming agent is not utilized during extraction.

Following extraction, the protein rich fraction may be separated from the slurry, for example, in a solid/liquid separation unit, consisting of a decanter and a disc-stack centrifuge. The protein rich fraction may be centrifuged at a low temperature, e.g., between 3-10° C. In some cases, the protein rich fraction is collected and the pellet is resuspended in, e.g., 3:1 water-to-protein. The process may be repeated, and the combined protein rich fractions filtered through a Nylon mesh.

### Starch Solids Separation

In some embodiments, the methods may optionally include reducing or removing a fraction comprising carbohydrates (e.g., starches) or a carbohydrate-rich protein isolate, post extraction.

### Charcoal Treatment

Optionally, the protein rich fraction, retentate fraction, or pulse protein isolate may be subjected to a carbon adsorption step to remove non-protein, off-flavor components, and additional fibrous solids from the protein extraction. This carbon adsorption step leads to a clarified protein extract. In one embodiment of a carbon adsorption step, the protein extract is then sent through a food-grade granular charcoal-filled annular basket column (<5% w/w charcoal-to-protein extract ratio) at 4 to 8° C.

### Ultrafiltration

The methods of the present disclosure may utilize ultrafiltration to separate the pulse proteins from other materials. The ultrafiltration process utilizes at least one semi-permeable selective membrane that separates a retentate fraction (containing materials that do not pass through the membrane) from a permeate fraction (containing materials that do pass through the membrane). The semi-permeable membrane separates materials (e.g., proteins and other components) based on molecular size. For example, the semi-permeable membrane used in the ultrafiltration processes of the present methods may exclude molecules (i.e., these molecules are retained in the retentate fraction) having a molecular size of 10 kDa or larger. In some embodiments, the semi-permeable membrane may exclude molecules (e.g., pulse proteins) having a molecular size of 25 kDa or larger. In some embodiments, the semi-permeable membrane excludes molecules having a molecular size of 50 kDa or larger. In various embodiments, the semi-permeable membrane used in the ultrafiltration process of the methods discussed herein excludes molecules (e.g., pulse proteins) having a molecular size greater than 5 kDa, 10 kDa, 15 kDa, 20 kDa, 25 kDa, 30 kDa, 35 kDa, 40, kDa, 45 kDa, 50 kDa, 55 kDa, 60 kDa, 65 kDa, 70 kDa, 75 kDa, 80 kDa, 85 kDa, 90 kDa, or 95 kDa. For example, a 10 kDa membrane allows molecules, including pulse proteins, smaller than 10 kDa in size to pass through the membrane into the permeate fraction, while molecules, including pulse proteins, equal to or larger than 10 kDa are retained in the retentate fraction. An exemplary protocol for the ultrafiltration process is provided in Example 1.

Ultrafiltration (UF) is a cross-flow separation process for separating compounds with particular molecular weights that are present in a liquid. By applying pressure, typically in the range of 20-500 psig to a membrane, the compounds having the specified molecular weight are separated from the liquid. UF membranes have molecular weight cut-off ranges of 1,000 to 500,000 Da. The pore sizes of the membranes typically range between 0.1 to 0.001 micron. The nominal pore size of a UF membrane with a 100 kD cut-off is typically about 0.006 micron and a membrane with a 10 kD cut-off is typically about 0.003 micron. If a liquid solution containing proteins, e.g., mung bean proteins, is subjected to ultrafiltration using a 10 kD membrane, the concentration of proteins having a molecular weight of less than 10 kD is increased in the filtrate (permeate) and decreased in the retentate. Concomitantly, the concentration of proteins having a molecular weight of greater than 10 kD is increased in the retentate and decreased in the filtrate (permeate). In various embodiments of the methods discussed herein, the semi-permeable membrane may have a pore size of 0.001, 0.0015, 0.002, 0.0025, 0.003, 0.0035, 0.004, 0.0045, 0.005, 0.0055, or 0.006 micron.

There are various types of UF membranes that are available commercially, including polymeric, ceramic, and metallic membranes having a desired molecular weight cutoff. For polymeric membrane types, these include membranes made from polyvinylidine fluoride (PVDF), polyether sulfone (PES), polyacrylonitrile (PAN), polytetrafluoroethylene (PTFE), polyamide-imide (PAI) and natural polymers including membranes made from rubber, wool, and cellulose. Metallic membranes are made by sintering metal powders onto a porous substrate. Commonly used metal powders are stainless steel, tungsten and palladium. Ceramic membranes are made of oxides, nitrides or carbides of metallic (e.g., aluminum and titanium) and non-metallic materials. UF membranes comprising zeolites are made of hydrated aluminosilicate minerals that contain alkali and alkaline-earth metals. Zeolite UF membranes are useful because of their highly uniform pore size.

The ultrafiltration process of the present methods may be performed at a temperature in a range of from about 5° C. to about 60° C. In some cases, the temperature may be about 15° C., about 20° C., about 25° C., about 30° C., about 35° C., about 40° C., about 45° C., or about 50° C. In some embodiments, the ultrafiltration process is performed at a pressure of from about 20 to about 500 psig. In various embodiments, the ultrafiltration process is performed at a pressure of about 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 psig.

### pH and Conductivity Adjustment

In some embodiments, the methods include adjusting the pH and/or conductivity of the retentate fraction or the pulse protein isolate. In some cases, the pH is adjusted to a range of from about 5.8 to about 6.6. In some embodiment, the pH is adjusted to from 6.0 to 6.2. In various embodiments, the pH is adjusted to 5.8, 5.9, 6.0, 6.1, 6.2, 6.3, 6.4, 6.5 or 6.6. In some embodiments, the conductivity of the retentate fraction or the pulse protein isolate is adjusted. In some embodiments, the conductivity of the retentate fraction or the pulse protein isolate is adjusted to between 1-3 mS/cm using salt if required. In various embodiments, the conductivity is adjusted to 1, 1.1, 1.2, 1.3, 1.4, 1.5, 1.6, 1.7, 1.8, 1.9, 2.0 2.1, 2.2, 2.3, 2.4, 2.5, 2.6, 2.7, 2.8, 2.9, or 3.0 mS/cm. In various embodiments, the salt used to modify the conductivity can be selected from sodium chloride, sodium sulfate, sodium phosphate, ammonium sulfate, ammonium phosphate, ammonium chloride, potassium chloride, potassium sulfate, or potassium phosphate. In some embodiments, the salt is NaCl.

In some embodiments, the methods include adjusting the pH and/or conductivity of the retentate fraction or the pulse protein isolate in two or more pH adjustment steps. In some cases, the pH is adjusted to a first pH range of from about 4.0 to about 6.6. Next, a second pH adjustment is made in which the pH of the retentate fraction or the pulse protein isolate is adjusted to be different, that is higher or lower, than the first pH of the retentate fraction or the pulse protein isolate. In some embodiments, the first pH adjustment is made to a pH of 4.0 to 6.0. In some embodiments, the pH achieved in the second pH adjustment is between 5.0 and 6.6. In various embodiments, the first pH is adjusted to 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0. In various embodiments, the second pH is adjusted to 5.0, 5.1, 5.2, 5.3, 5.4, 5.5, 5.6, 5.7, 5.8, 5.9, 6.0, 6.1, 6.2, 6.3, 6.4, 6.5 or 6.6. In various embodiments, the conductivity is adjusted to 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mS/cm. In various embodiments, the salt used to modify the conductivity can be selected from sodium chloride, sodium sulfate, sodium phosphate, ammonium sulfate, ammonium phosphate, ammonium chloride, potassium chloride, potassium sulfate, or potassium phosphate. In some embodiments, the salt is NaCl.

### Pasteurization and Drying

In some embodiments, the methods include heating the retentate fraction or the pulse protein isolate in a pasteurization process and/or drying the retentate fraction or the pulse protein isolate. In some embodiments, the retentate fraction or the pulse protein isolate is heated to a temperature of from about 70° C. to about 80° C. for a period of time (e.g., 20-30 seconds) to kill pathogens (e.g., bacteria). In a particular embodiment, pasteurization is performed at 74° C. for 20 to 23 seconds. In particular embodiments where a dry pulse protein isolate is desired, the pulse protein isolate may be passed through a spray dryer to remove any residual water content. The typical spray drying conditions include an inlet temperature of 170° C. and an outlet temperature of 70° C. The final dried protein isolate powder may comprise less than 10% or less than 5% moisture content.

### Order of Steps and Additional Steps

It is to be understood that the steps of the methods discussed above or herein may be performed in alternative orders consistent with the objective of producing a pulse protein isolate. In some embodiments, the methods may include additional steps, such as for example: recovering the purified protein isolate (e.g., using centrifugation), washing the purified protein isolate, making a paste using the purified protein isolate, or making a powder using the purified protein isolate. In some embodiments, the purified protein isolate is rehydrated (e.g., to about 80% moisture content), and the pH of the rehydrated purified protein isolate is adjusted to a pH of about 6. Unless otherwise noted, none of the embodiments discussed herein include isoelectric precipitation of the pulse proteins from a protein rich fraction (e.g., at a pH of from about 5 to about 6).

### Pulse Protein Isolates

The present disclosure includes pulse protein isolates (e.g., mung bean protein isolates), including those prepared by the methods discussed above. The pulse protein isolates are edible and comprise one or more desirable food qualities, including but limited to, high protein content, high protein purity, reduced retention of small molecular weight non-protein species (including mono and disaccharides), reduced retention of oils and lipids, superior structure building properties such as high gel strength and gel elasticity, superior sensory properties, and selective enrichment of highly functional 8 s globulin/beta conglycinin proteins.

In various embodiments, the pulse protein isolates provided herein are derived from dry beans, lentils, faba beans, dry peas, chickpeas, cowpeas, bambara beans, pigeon peas, lupins, vetches, adzuki, common beans, fenugreek, long beans, lima beans, runner beans, or tepary beans, soy beans, or mucuna beans. In various embodiments, the pulse protein isolates provided herein are derived from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius. In some embodiments, the pulse protein isolates are derived from mung beans. In some embodiments, the mung bean is Vigna radiata. In other embodiments, the milled composition may comprise almonds and other nuts, seeds such as sesame seeds, sunflower seeds, and other commonly consumed nuts, fruits and seeds. In various embodiments, the pulse protein isolate (e.g., mung bean protein isolate) discussed herein can be produced from any source of pulse protein (e.g., mung bean protein, including any varietal or cultivar of V. radiata). For example, the protein isolate can be prepared directly from whole plant material such as whole mung bean, or from an intermediate material made from the plant, for example, a dehulled bean, a non-dehulled bean, a flour, an air classified flour, a powder, a meal, ground grains, a cake (such as, for example, a defatted or de-oiled cake), or any other intermediate material suitable to the processing techniques disclosed herein to produce a pulse protein isolate. In some embodiments, the source of the plant protein may be a mixture of two or more intermediate materials. The examples of intermediate materials provided herein are not intended to be limiting.

### Characteristics of the Pulse Protein Isolates

In various embodiments, the pulse protein isolate (e.g., mung bean protein isolate) comprises pulse protein of from 50% to 60%, from 60% to 70%, from 70% to 80%, from 80% to 90%, or more. In some embodiments, the pulse protein isolate comprises 80%, 81%, 82%, 83%, 84%, 85%, 86%, 87%, 88%, 89%, 90%, 91%, 92%, 93%, 94%, 95%, 96%, 97%, 98%, or 99% or more pulse proteins. In some embodiments, at least 60% by weight of the pulse protein isolate is comprised of pulse proteins. In some embodiments, at least 65%, 70%, 75%, 80%, 85%, 90%, or 95% or more by weight of the pulse protein isolate comprises pulse proteins.

In some embodiments in which the pulse protein is mung bean protein, at least about 10%, 15%, 20%, 25%, 30%, 35%, 40%, 45%, 50%, 55%, 60%, 65%, 70%, 75%, 80%, 85% or greater than 85% by weight of the mung bean protein isolate consists of or comprises mung bean 8 s globulin/beta-conglycinin. In other embodiments, about 60% to 80%, 65% to 85%, 70% to 90%, or 75% to 95% by weight of the mung bean protein isolate consists of or comprises mung bean 8 s globulin/beta-conglycinin. In some embodiments, the mung bean protein isolate is reduced in the amount of lis globulin relative to whole mung bean or mung bean flour. In some embodiments, the amount of lis globulin is less than 10%, 8%, 8%, 7%, 6%, 5%, 4%, 3%, 2%, or 1% of the protein in the mung bean protein isolate.

In some embodiments, the pulse protein isolate (e.g., mung bean protein isolate) comprises about 1% to 10%, 2% to 9%, 3% to 8%, or 4% to 6% of carbohydrates (e.g., starch, polysaccharides, fiber) derived from the plant source of the isolate. In some embodiments, the pulse protein isolate comprises less than about 10%, 9%, 8%, 7%, 6% or 5% of carbohydrates derived from the plant source of the isolate. In some embodiments, the pulse protein isolate comprises about 9%, 8%, 7%, 6%, 5%, 4%, 3%, 2%, or about 1% of carbohydrates derived from the plant source of the isolate.

In some embodiments, the pulse protein isolate (e.g., mung bean protein isolate) comprises about 1% to 10%, 2% to 9%, 3% to 8%, or 4% to 6% of ash derived from the plant source of the isolate. In some embodiments, the pulse protein isolate comprises less than about 10%, 9%, 8%, 7%, 6% or 5% of ash derived from the plant source of the isolate. In some embodiments, the pulse protein isolate comprises about 9%, 8%, 7%, 6%, 5%, 4%, 3%, 2%, or about 1% of ash derived from the plant source of the isolate.

In some embodiments, the pulse protein isolate (e.g., mung bean protein isolate) comprises about 1% to 10%, 2% to 9%, 3% to 8%, or 4% to 6% of fats derived from the plant source of the isolate. In some embodiments, the pulse protein isolate comprises less than about 10%, 9%, 8%, 7%, 6% or 5% of fats derived from the plant source of the isolate. In some embodiments, the pulse protein isolate comprises about 9%, 8%, 7%, 6%, 5%, 4%, 3%, 2%, or about 1% of fats derived from the plant source of the isolate.

In some embodiments, the pulse protein isolate (e.g., mung bean protein isolate) comprises about 1% to 10% of moisture derived from the plant source of the isolate. In some embodiments, the pulse protein isolate comprises less than about 10%, 9%, 8%, 7%, 6% or 5% of moisture derived from the plant source of the isolate. In some embodiments, the pulse protein isolate comprises about 9%, 8%, 7%, 6%, 5%, 4%, 3%, 2%, or about 1% of moisture derived from the plant source of the isolate.

In various embodiments, the pulse protein isolate (e.g., mung bean protein isolate) comprises pulse proteins having a molecular size of less than 100 kilodaltons (kDa). In some embodiments, the pulse protein isolate comprises pulse proteins having a molecular size of less than 95 kDa, 90 kDa, 85 kDa, 80 kDa, 75 kDa, 70 kDa, 65 kDa, 60, kDa, 55 kDa, 50 kDa, 45 kDa, 40 kDa, 35 kDa, 30 kDa, 25 kDa, 20 kDa or 15 kDa. In various embodiments, the pulse protein isolate comprises pulse proteins having a molecular size of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa. Unless otherwise noted, references to a pulse protein isolate comprising pulse proteins having a specified molecular weight does not exclude the possibility that the same pulse protein isolate or retentate fraction also contains pulse proteins of other molecular weights.

In various embodiments, the pulse protein isolate (e.g., mung bean protein isolate) comprises pulse proteins enriched in proteins having a molecular size of greater than 5 kilodaltons (kDa). In some embodiments, the pulse protein isolate comprises pulse proteins enriched in proteins having a molecular size of greater than 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa or 95 kDa. In some embodiments, the pulse protein isolated comprises pulse proteins enriched in proteins having a molecular size of less than 100 kDa. In some embodiments, the pulse protein isolate (e.g., mung bean protein isolate) comprises pulse proteins enriched in proteins having a molecular size of from 1 kDa to 99 kDa, from 1 kDa to 75 kDa, from 1 kDa to 50 kDa, from 1 kDa to 25 kDa, from 5 kDa to 99 kDa, from 5 kDa to 75 kDa, from 5 kDa to 50 kDa, from 5 kDa to 25 kDa, from 10 kDa to 99 kDa, from 10 kDa to 75 kDa, from 10 kDa to 50 kDa, or from 10 kDa to 25 kDa. In various embodiments, the pulse protein isolate comprises, or is enriched in, pulse proteins having a molecular size of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa. Unless otherwise noted, references to a pulse protein isolate (or retentate fraction) comprising pulse proteins having a specified molecular weight does not exclude the possibility that the same pulse protein isolate or retentate fraction also contains pulse proteins of other molecular weights.

In various embodiments, the pulse protein isolate (e.g., mung bean protein isolate) comprises pulse proteins depleted in proteins having a molecular size of less than 5 kilodaltons (kDa). In some embodiments, the pulse protein isolate comprises pulse proteins depleted in proteins having a molecular size of less than 10 kDa, 15 kDa, 20 kDa, 25 kDa, 30 kDa, 40 kDa, 45 kDa, 50 kDa, 55 kDa, 60 kDa, 65 kDa, 70 kDa, 80 kDa, 85 kDa, 95 kDa or 95 kDa. In various embodiments, the pulse protein isolate comprises, or is enriched in, pulse proteins having a molecular size of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa. Unless otherwise noted, references to a pulse protein isolate comprising pulse proteins having a specified molecular weight does not exclude the possibility that the same pulse protein isolate or retentate fraction also contains pulse proteins of other molecular weights.

### Reduced Allergen, Anti-Nutritional, and Environmental Contaminant Content

In some embodiments, the pulse protein isolates (e.g., mung bean protein isolate) provided herein have a reduced allergen content. In some embodiments, the reduced allergen content is relative to the allergen content of the plant source of the isolate. The pulse protein isolate or a composition comprising the pulse protein isolate may be animal-free, dairy-free, soy-free and gluten-free. Adverse immune responses such as hives or rash, swelling, wheezing, stomach pain, cramps, diarrhea, vomiting, dizziness and even anaphylaxis presented in subjects who are typically allergic to eggs may be averted. Further, the pulse protein isolate or a composition comprising the pulse protein isolate may not trigger allergic reactions in subjects based on milk, eggs, soy and wheat allergens. Accordingly, in some embodiments, the pulse protein isolate or a composition comprising the pulse protein isolate is substantially free of allergens.

Dietary anti-nutritional factors are chemical substances that can adversely impact the digestibility of protein, bioavailability of amino acids and protein quality of foods (Gilani et al., 2012). In some embodiments, the pulse protein isolates (e.g., mung bean protein isolates) provided herein have reduced amounts of anti-nutritional factors. In some embodiments, the reduced amount of anti-nutritional factors is relative to the content of the plant source of the isolate. In some embodiments, the reduced anti-nutritional factor is selected from the group consisting of tannins, phytic acid, hemagglutinins (lectins), polyphenols, trypsin inhibitors, α-amylase inhibitors, lectins, protease inhibitors, and combinations thereof.

In various embodiments, environmental contaminants are either free from the pulse protein isolates (e.g., mung bean protein isolates), below the level of detection of 0.1 ppm, or present at levels that pose no toxicological significance. In some embodiments, the reduced environmental contaminant is a pesticide residue. In some embodiments, the pesticide residue is selected from the group consisting of: chlorinated pesticides, including alachlor, aldrin, alpha-BHC, alpha-chlordane, beta-BHC, DDD, DDE, DDT, delta-BHC, dieldrin, endosulfan I, endosulfan II, endosulfan sulfate, endrin, endrin aldehyde, gamma-BHC, gamma-chlordane, heptachlor, heptachlor epoxide, methoxyclor, and permethrin; and organophosphate pesticides including azinophos methyl, carbophenothion, chlorfenvinphos, chlorpyrifos methyl, diazinon, dichlorvos, dursban, dyfonate, ethion, fenitrothion, malathion, methidathion, methyl parathion, parathion, phosalone, pirimiphos methyl, and combinations thereof. In some embodiments, the reduced environmental contaminant is selected from residues of dioxins and polychlorinated biphenyls (PCBs), or mycotoxins such as aflatoxin B1, B2, G1, G2, and ochratoxin A.

### Other Food Functionality Characteristics of the Pulse Protein Isolates

In various embodiments, the pulse protein isolates (e.g., mung bean protein isolates) exhibit desirable functional characteristics such as emulsification, water binding, foaming and gelation properties comparable to an egg. In various embodiments, the pulse protein isolates exhibit one or more functional properties advantageous for use in food compositions. The functional properties may include, but are not limited to, crumb density, structure/texture, elasticity/springiness, coagulation, binding, moisturizing, mouthfeel, leavening, aeration/foaming, creaminess, and emulsification of the food composition. Mouthfeel is a concept used in the testing and description of food products. Products made using pulse protein isolates discussed herein can be assessed for mouthfeel. Products, e.g., baked goods, made using the pulse protein isolates have mouthfeel that is similar to products made with natural eggs. In some embodiments the mouthfeel of the products made using the pulse protein isolates is superior to the mouthfeel of previously known or attempted egg substitutes, e.g., bananas, modified whey proteins, or Egg Beaters™.

Examples of properties which may be included in a measure of mouthfeel include: Cohesiveness: degree to which the sample deforms before rupturing when biting with molars; Density: compactness of cross section of the sample after biting completely through with the molars; Dryness: degree to which the sample feels dry in the mouth; Fracturability: force with which the sample crumbles, cracks or shatters (fracturability encompasses crumbliness, crispiness, crunchiness and brittleness); Graininess: degree to which a sample contains small grainy particles, may be seen as the opposite of smoothness; Gumminess: energy required to disintegrate a semi-solid food to a state ready for swallowing; Hardness: force required to deform the product to given distance, i.e., force to compress between molars, bite through with incisors, compress between tongue and palate; Heaviness: weight of product perceived when first placed on tongue; Moisture absorption: amount of saliva absorbed by product; Moisture release: amount of wetness/juiciness released from sample; Mouthcoating: type and degree of coating in the mouth after mastication (for example, fat/oil); Roughness: degree of abrasiveness of product's surface perceived by the tongue; Slipperiness: degree to which the product slides over the tongue; Smoothness: absence of any particles, lumps, bumps, etc., in the product; Uniformity: degree to which the sample is even throughout; homogeneity; Uniformity of Bite: evenness of force through bite; Uniformity of Chew: degree to which the chewing characteristics of the product are even throughout mastication; Viscosity: force required to draw a liquid from a spoon over the tongue; and Wetness: amount of moisture perceived on product's surface.

The pulse protein isolates discussed herein may also have one or more functional properties alone or when incorporated into a food composition. Such functional properties may include, but are not limited to, one or more of emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, or color. In some embodiments, at least one functional property of the pulse protein isolate differs from the corresponding functional property of the source of the plant protein. In some embodiments, at least one functional property of the pulse protein isolate (alone or when incorporated into a food composition) is similar or equivalent to the corresponding functional property of a reference food product, such as, for example, an egg (liquid, scrambled, or in patty form), a cake (e.g., pound cake, yellow cake, or angel food cake), a cream cheese, a pasta, an emulsion, a confection, an ice cream, a custard, milk, a deli meat, chicken (e.g., chicken nuggets), or a coating. In some embodiments, the pulse protein isolate, either alone or when incorporated into a composition, is capable of forming a gel under heat or at room temperature.

### Modified Organoleptic Properties

The pulse protein isolates discussed herein may have modulated organoleptic properties of one or more of the following characteristics: astringent, beany, bitter, burnt, buttery, nutty, sweet, sour, fruity, floral, woody, earthy, beany, spicy, metallic, sweet, musty, grassy, green, oily, vinegary, neutral and bland flavor or aromas. In some embodiments, the pulse protein isolates exhibit modulated organoleptic properties such as a reduction or absence in one or more of the following: astringent, beany, bitter, burnt, buttery, nutty, sweet, sour, fruity, floral, woody, earthy, beany, spicy, metallic, sweet, musty, grassy, green, oily, vinegary neutral and bland flavor or aromas.

In some cases, methods to reduce or remove at least one impurity that may impart or is associated with an off-flavor or off-odor in the pulse protein isolate may be undertaken. The one or more impurity may be a volatile or nonvolatile compound and may comprise, for example, lipoxygenase, which is known to catalyze oxidation of fatty acids. In other cases, the at least one impurity may comprise a phenol, an alcohol, an aldehyde, a sulfide, a peroxide, or a terpene. Other biologically active proteins classified as albumins may also be removed, including lectins and protease inhibitors such as serine protease inhibitors and tryptic inhibitors. In some embodiments, impurities are reduced by a solid absorption procedure using, for example, charcoal, a bentonite clay, or activated carbon.

In some embodiments, the at least one impurity may comprise one or more substrates for an oxidative enzymatic activity, for example one or more fatty acids. In some embodiments, the pulse protein isolates contain reduced amounts of one or more fatty acids selected from: C14:0 (methyl myristate); C15:0 (methyl pentadecanoate); C16:0 (methyl palmitate; C16:1 methyl palmitoleate; C17:0 methyl heptadecanoate; C18:0 methyl stearate; C18:1 methyl oleate; C18:2 methyl linoleate; C18:3 methyl alpha linoleate; C20:0 methyl eicosanoate; and C22:0 methyl behenate to reduce rancidity.

In some embodiments, the pulse protein isolate (e.g., mung bean protein isolate) has a reduced oxidative enzymatic activity relative to the source of the pulse protein. For example, a purified mung bean isolate can have about a 5%, 10%, 15%, 20%, or 25% reduction in oxidative enzymatic activity relative to the source of the mung bean protein. In some embodiments, the oxidative enzymatic activity is lipoxygenase activity. In some embodiments, the pulse protein isolate has lower oxidation of lipids or residual lipids relative to the source of the plant protein due to reduced lipoxygenase activity.

In additional embodiments, reducing the at least one impurity comprises removing a fibrous solid, a salt, or a carbohydrate. Reducing such impurity comprises removing at least one compound that may impart or is associated with the off-flavor or off-odor. Such compounds may be removed, for example, using an activated charcoal, carbon, or clay. As another example, the at least one compound may be removed using a chelating agent (e.g., EDTA, citric acid, or a phosphate) to inhibit at least one enzyme that oxidizes a lipid or a residual lipid. In a particular example, EDTA may be used to bind co-factor for lipoxygenase, an enzyme that can oxidize residual lipid to compounds, e.g. hexanal, which are known to leave to off-flavors.

### Food Compositions Containing Pulse Protein Isolates

The pulse protein isolates (e.g., mung bean protein isolates) discussed herein may be incorporated into a food composition along with one or more other edible ingredients. In some cases, the pulse protein isolate may be used as a direct protein replacement of animal- or vegetable-based protein in a variety of conventional food and beverage products across multiple categories. In some embodiments, the use levels range from 3 to 90% w/w of the final product. Exemplary food compositions in which the pulse protein isolates can be used are discussed below. In some embodiments, the pulse protein isolate is used as a supplement to existing protein in food products. In any of the various embodiments of the food compositions, the pulse protein isolate may be contacted with a cross-linking enzyme to cross-link the pulse proteins. In various embodiments, the cross-linking enzyme is selected from transglutaminase, sortase, subtilisin, tyrosinase, laccase, peroxidase, or lysyl oxidase. In some embodiments, the cross-linking enzyme is transglutaminase. In any of the various embodiments of the food compositions, the pulse protein isolate may be contacted with a protein modifying enzyme such as papain, pepsin, rennet, coagulating enzymes or sulfhydryl oxidase to modify the structure of the pulse proteins.

The pulse protein isolates provided herein are suitable for various food applications and can be incorporated into, e.g., edible egg-free emulsion, egg analog, egg-free scrambled eggs, egg-free patty, egg-free pound cake, egg-free angel food cake, egg-free yellow cake, egg-free cream cheese, egg-free pasta dough, egg-free custard, egg-free ice cream, and dairy-free milk. The pulse protein isolates can also be used as replacement ingredients in various food applications including but not limited to meat substitutes, egg substitutes, baked goods and fortified drinks

In various embodiments, one or more pulse protein isolates can be incorporated into multiple food compositions, including liquid and patty scrambled egg substitutes to a desired level of emulsification, water binding and gelation. In an embodiment, a functional egg replacement product comprises pulse protein isolate (8-15%), and one or more of: oil (10%), hydrocolloid, preservative, and optionally flavors, water, lecithin, xanthan, sodium carbonate, and black salt.

In some embodiments, the pulse protein isolate is incorporated in an egg substitute composition. In some such embodiments, the organoleptic property of the pulse protein isolate (e.g., a flavor or an aroma) is similar or equivalent to a corresponding organoleptic property of an egg. The egg substitute composition may exhibit at least one functional property (e.g., emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, or color) that is similar or equivalent to a corresponding functional property of an egg. In addition to the pulse protein isolate, the egg substitute composition may include one or more of iota-carrageenan, gum arabic, konj ac, xanthan gum, or gellan.

In some embodiments, the pulse protein isolate is incorporated in an egg-free cake, such as a pound cake, a yellow cake, or an angel food cake. In some such embodiments, at least one organoleptic property (e.g., a flavor or an aroma) of the egg-free cake is similar or equivalent to a corresponding organoleptic property of a cake containing eggs. The egg-free cake may exhibit at least one functional property similar or equivalent to a corresponding functional property of a cake containing eggs. The at least one function property may be, for example, one or more of emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, color, or a combination thereof. In some embodiments in which the pulse protein isolate is included in an egg-free pound cake, a peak height of the egg-free pound cake is at least 90% of the peak height of a pound cake containing eggs.

In some embodiments, the pulse protein isolate is incorporated into an egg-free cake mix or an egg-free cake batter. In some such embodiments, the egg-free cake mix or batter has at least one organoleptic property (e.g., a flavor or aroma) that is similar or equivalent to a corresponding organoleptic property of a cake mix or batter containing eggs. The egg-free cake mix or batter may exhibit at least one functional property similar or equivalent to a corresponding functional property of a cake batter containing eggs. The at least one functional property may be, for example, one or more of emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, color, or a combination thereof. In some embodiments in which the pulse protein isolate is included in an egg-free pound cake batter, a specific gravity of the egg-free pound cake batter is 0.95-0.99.

In some cases, increased functionality is associated with the pulse protein isolate in a food composition. For instance, food products produced with the pulse protein isolates discussed herein may exhibit increased functionality in dome or crack, cake resilience, cake cohesiveness, cake springiness, cake peak height, specific gravity of batter, center doming, center crack, browning, mouthfeel, spring-back, off flavors or flavor.

In some embodiments, the pulse protein isolate is included in a cream cheese, a pasta dough, a pasta, a milk, a custard, a frozen dessert (e.g., a frozen dessert comprising ice cream), a deli meat, or chicken (e.g., chicken nuggets).

In some embodiments, the pulse protein isolate is incorporated into a food or beverage composition, such as, for example, an egg substitute, a cake (e.g., a pound cake, a yellow cake, or an angel food cake), a cake batter, a cake mix, a cream cheese, a pasta dough, a pasta, a custard, an ice cream, a milk, a deli meat, or a confection. The food or beverage composition may provide sensory impressions similar or equivalent to the texture and mouthfeel that replicates a reference food or beverage composition. In some embodiments, before being included in a food or beverage composition, the pulse protein isolate is further processed in a manner that depends on a target application for the pulse protein isolate. For example, the pulse protein isolate may be diluted in a buffer to adjust the pH to a pH appropriate for the target application. As another example, the pulse protein isolate may be concentrated for use in the target application. As yet another example, the pulse protein isolate may be dried for use in the target application. Various examples of food compositions comprising the pulse protein isolates discussed herein are provided below.

### Scrambled Egg Analog Using Transglutaminase

In some embodiments, the pulse protein isolates are incorporated into a scrambled egg analog in which the pulse protein isolate (e.g., mung bean protein isolate) has been contacted with transglutaminase (or other cross-linking enzyme) to provide advantageous textural, functional and organoleptic properties. Food processing methods employing transglutaminases are known in the art.

In some embodiments, the transglutaminase is microencapsulated when utilized in the egg analogs provided herein. Microencapsulation of transglutaminase enzyme in such egg mimetic emulsions maintains a stable emulsion by preventing contact of the protein substrate with the transglutaminase enzyme. A cross-linking reaction is initiated upon heating to melt the microencapsulating composition. In some embodiments, the transglutaminase is immobilized on inert porous beads or polymer sheets, and contacted with the egg mimetic emulsions.

In certain aspects of the invention, the method for producing an egg substitute composition comprises contacting a pulse protein isolate with an amount of transglutaminase, preferably between 0.0001% to 0.1%. In some embodiments, the method provides an amount of transglutaminase between 0.001% and 0.05%. In some embodiments, the method provides an amount of transglutaminase between 0.001% and 0.0125%.

In various embodiments, the scrambled egg analog comprises a pulse protein isolate described herein, along with one or more of the following components: water, disodium phosphate and oil. In some embodiments, the scrambled egg analog further comprises NaCl. In some embodiments, the scrambled egg analog has been contacted with transglutaminase. In a particular embodiment, the scrambled egg analog comprises: Protein Solids: 11.3 g, Water: 81.79 g, Disodium phosphate: 0.4 g, Oil: 6.2 g, NaCl: 0.31 g (based on total weight of 100 g) wherein the protein solids are contacted with between 0.001% and 0.0125% of transglutaminase.

In some embodiments, the composition lacks lipoxygenase.

### Vegan Patty

Pulse protein isolates (e.g., mung bean protein isolates) can be used as the sole gelling agent in a formulated vegan patty. In some embodiments, a hydrocolloid system comprised of iota-carrageenan and gum arabic enhances native gelling properties of the pulse protein isolate in a formulated patty. In other embodiments, a hydrocolloid system comprised of high-acyl and low-acyl gellan in a 1.5:1 ratio enhances native gelling properties of the pulse protein isolate in a formulated patty. In further embodiments, a hydrocolloid system comprised of konjac and xanthan gum enhances native gelling properties of the pulse protein isolate in a formulated patty.

### Egg-Free Emulsion

In another embodiment, pulse protein isolates (e.g., mung bean protein isolates) are included in an edible egg-free emulsion. In some embodiments, the emulsion comprises one or more additional components selected from water, oil, fat, hydrocolloid, and starch. In some embodiments, at least or about 60-85% of the edible egg-free emulsion is water. In some embodiments, at least or about 10-20% of the edible egg-free emulsion is the pulse protein isolate. In some embodiments, at least or about 5-15% of the edible egg-free emulsion is oil or fat. In some embodiments, at least or about 0.01-6% of the edible egg-free emulsion is the hydrocolloid fraction or starch. In some embodiments, the hydrocolloid fraction comprises high-acyl gellan gum, low-acyl gellan gum, iota-carrageenan, gum arabic, konjac, locust bean gum, guar gum, xanthan gum, or a combination of one or more gums thereof. In some embodiments, the emulsion further comprises one or more of: a flavoring, a coloring agent, an antimicrobial, a leavening agent, and salt. In some embodiments, the emulsion further comprises phosphate.

In an embodiment, the edible egg-free emulsion has a pH of about 5.6 to 6.8. In some cases, the edible egg-free emulsion comprises water, a pulse protein isolate as described herein, an enzyme that modifies a structure of the protein isolate, and oil or fat. In some embodiments, the enzyme comprises a transglutaminase or proteolytic enzyme. In some embodiments, at least or about 70-85% of the edible egg-free emulsion is water. In some embodiments, at least or about 7-15% of the edible egg-free emulsion is the pulse protein isolate. In some embodiments, at least or about 0.0005-0.0025% (5-25 parts per million) of the edible egg-free emulsion is the enzyme that modifies the structure of the pulse protein isolate. In some embodiments, at least or about 5-15% of the edible egg-free emulsion is oil or fat.

### Baked Cake Mixes and Batters

In another embodiment, pulse protein isolates (e.g., mung bean protein isolates) are included in one or more egg-free cake mixes, suitable for preparing one or more egg-free cake batters, from which one or more egg-free cakes can be made. In some embodiments, the egg-free cake mix comprises flour, sugar, and a pulse protein isolate. In some embodiments, the egg-free cake mix further comprises one or more additional components selected from: cream of tartar, disodium phosphate, baking soda, and a pH stabilizing agent. In some embodiments, the flour comprises cake flour.

In another embodiment, pulse protein isolates (e.g., mung bean protein isolates) are included in an egg-free cake batter comprising an egg-free cake mix described above, and water. In some embodiments, the egg-free cake batter is an egg-free pound cake batter, an egg-free angel food cake batter, or an egg-free yellow cake batter. In some embodiments, the egg-free cake batter has a specific gravity of 0.95-0.99.

In an embodiment, an egg-free pound cake mix comprises flour, sugar, and a pulse protein isolate. In some embodiments, the flour comprises cake flour. In some embodiments, the egg-free pound cake mix further comprises oil or fat. In some embodiments, the oil or fat comprises butter or shortening. In some embodiments, at least or about 25-31% of the egg-free pound cake batter is flour. In some embodiments, at least or about 25-31% of the egg-free pound cake batter is oil or fat. In some embodiments, at least or about 25-31% of the egg-free pound cake batter is sugar. In some embodiments, at least or about 6-12% of the egg-free pound cake batter is the pulse protein isolate. In some embodiments, the batter further comprises disodium phosphate or baking soda.

In an embodiment, an egg-free pound cake batter comprises an egg-free pound cake mix described above, and further comprises water. In some embodiments, the egg-free pound cake batter comprises about four parts of the egg-free pound cake mix; and about one part water. In some embodiments, at least or about 20-25% of the egg-free pound cake batter is flour. In some embodiments, at least or about 20-25% of the egg-free pound cake batter is oil or fat. In some embodiments, at least or about 20-25% of the egg-free pound cake batter is sugar. In some embodiments, at least or about 5-8% of the egg-free pound cake batter is the pulse protein isolate. In some embodiments, at least or about 18-20% of the egg-free pound cake batter is water.

In an embodiment, an egg-free angel food cake mix comprises flour, sugar, and a pulse protein isolate. In some embodiments, at least or about 8-16% of the egg-free angel food cake mix is flour. In some embodiments, at least or about 29-42% of the egg-free angel food cake mix is sugar. In some embodiments, at least or about 7-10% of the egg-free angel food cake mix is the pulse protein isolate. In some embodiments, the egg-free angel food cake mix further comprises cream of tartar, disodium phosphate, baking soda, or a pH stabilizing agent. In some embodiments, the flour comprises cake flour. Also provided herein is an egg-free angel food cake batter comprising an egg-free angel food cake mix described above, and water.

In an embodiment, an egg-free yellow cake mix comprises flour, sugar, and a pulse protein isolate. In some embodiments, at least or about 20-33% of the egg-free yellow cake mix is flour. In some embodiments, at least or about 19-39% of the egg-free yellow cake mix is sugar. In some embodiments, at least or about 4-7% of the egg-free yellow cake mix is the pulse protein isolate. In some embodiments, the egg-free yellow cake mix further comprises one or more of baking powder, salt, dry milk, and shortening. Also provided herein is an egg-free yellow cake batter comprising an egg-free yellow cake mix described above, and water.

Sensory quality parameters of cakes made with the pulse protein isolates are characterized as fluffy, soft, airy texture. The peak height is measured to be 90-110% of pound cake containing eggs. The specific gravity of cake batter with the purified pulse protein isolate is 0.95-0.99, similar to that of cake batter with whole eggs of 0.95-0.96.

### Cream Cheese Analog

In another embodiment, pulse protein isolates (e.g., mung bean protein isolates) are included in an egg-free cream cheese. In some embodiments, the egg-free cream cheese comprises one or more additional components selected from water, oil or fat, and hydrocolloid. In some embodiments, at least or about 75-85% of the egg-free cream cheese is water. In some embodiments, at least or about 10-15% of the egg-free cream cheese is the pulse protein isolate. In some embodiments, at least or about 5-10% of the egg-free cream cheese is oil or fat. In some embodiments, at least or about 0.1-3% of the egg-free cream cheese is hydrocolloid. In some embodiments, the hydrocolloid comprises xanthan gum or a low-methoxy pectin and calcium chloride system. In some embodiments, the egg-free cream cheese further comprises a flavoring or salt. In some embodiments, one or more characteristics of the egg-free cream cheese is similar or equivalent to one or more corresponding characteristics of a cream cheese containing eggs. In some embodiments, the characteristic is a taste, a viscosity, a creaminess, a consistency, a smell, a spreadability, a color, a thermal stability, or a melting property. In some embodiments, the characteristic comprises a functional property or an organoleptic property. In some embodiments, the functional property comprises: emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, or color. In some embodiments, the organoleptic property comprises a flavor or an odor.

### Egg-Free Pasta Dough

In another embodiment, pulse protein isolates (e.g., mung bean protein isolates) are included in an egg-free pasta dough. In some embodiments, the egg-free pasta dough comprises one or more additional components selected from flour, oil or fat, and water. In some embodiments, the flour comprises semolina flour. In some embodiments, the oil or fat comprises extra virgin oil. In some embodiments, the egg-free pasta dough further comprises salt. Also provided herein is an egg-free pasta made from an egg-free pasta dough described above. In some embodiments, the egg-free pasta is fresh. In some embodiments, the egg-free pasta is dried. In some embodiments, one or more characteristics of the egg-free pasta is similar or equivalent to one or more corresponding characteristics of a pasta containing eggs. In some embodiments, the one or more characteristics comprise chewiness, density, taste, cooking time, shelf life, cohesiveness, or stickiness. In some embodiments, the one or more characteristics comprise a functional property or an organoleptic property. In some embodiments, the functional property comprises: emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, or color. In some embodiments, the organoleptic property comprises a flavor or an odor.

### Plant-Based Milk

In another embodiment, pulse protein isolates (e.g., mung bean protein isolates) are included in a plant-based milk. In some embodiments, the plant-based milk comprises one or more additional components selected from water, oil or fat, and sugar. In some embodiments, at least or about 5% of the plant-based milk is the pulse protein isolate. In some embodiments, at least or about 70% of the plant-based milk is water. In some embodiments, at least or about 2% of the plant-based milk is oil or fat. In some embodiments, the plant-based milk further comprises one or more of: disodium phosphate, soy lecithin, and trace minerals. In particular embodiments, the plant-based milk is lactose-free. In other particular embodiments, the plant-based milk does not comprise gums or stabilizers.

### Egg-Free Custard

In another embodiment, pulse protein isolates (e.g., mung bean protein isolates) are included in an egg-free custard. In some embodiments, the egg-free custard comprises one or more additional components selected from cream and sugar. In some embodiments, at least or about 5% of the egg-free custard is the pulse protein isolate. In some embodiments, at least or about 81% of the egg-free custard is cream. In some embodiments, at least or about 13% of the egg-free custard is sugar. In some embodiments, the egg-free custard further comprises one or more of: iota-carrageenan, kappa-carrageenan, vanilla, and salt. In some embodiments, the cream is heavy cream.

### Egg-Free Ice Cream

In another embodiment, pulse protein isolates (e.g., mung bean protein isolates) are included in an egg-free ice cream. In some embodiments, the egg-free ice cream is a soft-serve ice cream or a regular ice cream. In some embodiments, the egg-free ice cream comprises one or more additional components selected from cream, milk, and sugar. In some embodiments, at least or about 5% of the egg-free ice cream is the protein isolate. In some embodiments, at least or about 41% of the egg-free ice cream is cream. In some embodiments, at least or about 40% of the egg-free ice cream is milk. In some embodiments, at least or about 13% of the egg-free ice cream is sugar. In some embodiments, the egg-free ice cream further comprises one or more of iota carrageenan, kappa carrageenan, vanilla, and salt. In some embodiments, the cream is heavy cream. In some embodiments, the milk is whole milk. In particular embodiments, the egg-free ice cream is lactose-free. In some embodiments, the egg-free ice cream does not comprise gums or stabilizers. In some embodiments, the egg-free ice provides a traditional mouthfeel and texture of an egg-based ice cream but melts at a slower rate relative to an egg-based ice cream.

### Fat Reduction Shortening System (FRSS)

In another embodiment, pulse protein isolates (e.g., mung bean protein isolates) are included in a fat reduction shortening system. In some embodiments, the FRSS comprises one or more additional components selected from water, oil or fat. In some embodiments, the FRSS further comprises sodium citrate. In further some embodiments, the FRSS further comprises citrus fiber. In some embodiments, at least or about 5% of the FRSS is the pulse protein isolate. In preferred embodiments, the pulse protein-based FRSS enables a reduction in fat content in a food application (e.g., a baking application) utilizing the FRSS, when compared to the same food application utilizing an animal and/or dairy based shortening. In some embodiments, the reduction in fat is at least 10%, 20%, 30% or 40% when compared to the same food application utilizing an animal and/or dairy based shortening.

### Meat Analogues

In another embodiment, pulse protein isolates (e.g., mung bean protein isolates) are included in a meat analogue. In some embodiments, the meat analogue comprises one or more additional components selected from water, oil, disodium phosphate, transglutaminase, starch and salt. In some embodiments, at least or about 10% of the meat analogue is the pulse protein isolate. In some embodiments, preparation of the meat analogue comprises mixing the components of the meat analogue into an emulsion and pouring the emulsion into a casing that can be tied into a chubb. In some embodiments, chubs containing the meat analogue are incubated in a water bath at 50° C. for 2 hours. In further embodiments, the incubated chubbs are pressure cooked. In some embodiments, the pressure cooking occurs at 15 psi at about 121° C. for 30 minutes.

### Food Applications: Co-Ingredients

Various gums, phosphates, starches, preservatives, and other ingredients may be included in the food compositions comprising the pulse protein isolates.

Various gums useful for formulating one or more pulse protein based food products described herein include, e.g., konjac, gum acacia, Versawhip, Guar+Xanthan, Q-extract, CMC 6000 (Carboxymethylcellulose), Citri-Fi 200 (citrus fiber), Apple fiber, Fenugreek fiber.

Various phosphates useful for formulating one or more pulse protein based food products described herein include disodium phosphate (DSP), sodium hexamethaphosphate (SHMP), and tetrasodium pyrophosphate (TSPP).

Starch may be included as a food ingredient in the pulse protein food products described herein. Starch has been shown to have useful emulsifying properties; starch and starch granules are known to stabilize emulsions. Starches are produced from plant compositions, such as, for example, arrowroot starch, cornstarch, tapioca starch, mung bean starch, potato starch, sweet potato starch, rice starch, sago starch, wheat starch.

In certain embodiments, the food compositions comprise an effective amount of an added preservative in combination with the pulse protein isolate. The preservative may include ascorbic acid, citric acid, sodium benzoate, calcium propionate, sodium erythorbate, sodium nitrite, calcium sorbate, potassium sorbate, BHA, BHT, EDTA, tocopherols (Vitamin E) or antioxidants.

### Storage And Shelf Life of Food Compositions

In some embodiments, the food compositions comprising the pulse protein isolates may be stable in storage at room temperature for up to 1, 2, 3, 4, 5, 6, 7, 8, 9, or 10 weeks. In some embodiments, the food compositions comprising the pulse protein isolates may be stable for storage at room temperature for months, e.g., greater than 1, 2, 3, 4, 5, 6, 7, 8, 9, 10, 11, 12, or 13 months. In some embodiments, the food compositions comprising the pulse protein isolates may be stable for refrigerated or freezer storage for months, e.g., greater than 1, 2, 3, 4, 5, 6, 7, 8, 9, 10, 11, 12, or 13 months. In some embodiments, the food compositions comprising the pulse protein isolates may be stable for refrigerated or freezer storage for years, e.g., greater than 1, 2, 3, 4, 5, 6, 7, 8, 9, 10, 11, 12, or 13 years.

In some embodiments, storage as a dry material can increase the shelf-life of the pulse protein isolate or a food composition comprising the pulse protein isolate. In some embodiments, the pulse protein isolate or a food composition comprising the pulse protein isolate is stored as a dry material for later reconstitution with a liquid, e.g., water. In some embodiments, the pulse protein isolate or the food composition is in powdered form, which may be less expensive to ship, lowers risk for spoilage and increases shelf-life (due to greatly reduced water content and water activity).

In various embodiments, a food composition (e.g., an egg-free liquid egg analog product) comprising the pulse protein isolate has a viscosity of less than 500 cP after storage for thirty days at 4° C. In some cases, the composition has a viscosity of less than 500 cP after storage for sixty days at 4° C. In various embodiments, a food composition (e.g., an egg-free liquid egg analog product) comprising the pulse protein isolate has a viscosity of less than 450 cP after storage for thirty days at 4° C. In some cases, the composition has a viscosity of less than 450 cP after storage for sixty days at 4° C.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Heat Treatment of a Pulse and Manufacture of Heat Treated Pulses

Mung beans were purchased from a commercial source and heat treated. Mung beans were heat treated in a RevTech machine (Revtech, PA Champgrand, 50 allée des abricotiers, 26270 Loriol Sur Drome, France). The mung beans were continuously moved through the stainless-steel vertical spiral tubes assisted with vibrational movement of the unit. The vertical angle and oscillation frequency of the vertical vibratory tubes determines the speed of the mung beans traveling through the tubes. The beans are fed from the bottom and travel upwards through the tubes and were discharged from the top outlet into a fluidized bed cooler (cooling zone) where the beans are cooled down.

The spiral tubes can be divided into various heating zones for differential heat treatment as the mung beans travels upward through the tubes. The residence time in each zone can be controlled based on the speed and number of tubes selected for each heating zone.

The heat treatment was divided into two heating zones (zone 1 and 2) in the vertical vibratory tubes and one cooling zone (zone 3) in the fluidized bed cooler. The zone 1 temperature was kept between 100° C. to 150° C. and steam was added at 5% by weight basis of the beans bein fend into the dryer per hour. The steam was generated through a standard boiler installation and injected through through connecting tubes directly into zone 1 of the RevTech machine. The zone 2 temperature was kept between 140° C. to 225° C. The residence time through zone 1 was 3 minutes and through zone 2 was 3 minutes, for total heat treatment of 6 min. After the beans passed through both heating zones, the beans were conveyed into the fluidized bed dryer (cooling zone) and cooled down to 30° C.-50° C.

Heat treatment of pulses was also performed in batch mode or a continuous mode in a fluidized bed dryer. In this heat treatment regime, the beans were exposed to heat and steam treatment in the fluidized bed dryer. In batch mode operation, the mung beans were fed into the fluidized bed dryer at a rate of between 1 to 20 kg/hr on a metal screen bed with or without shaking motion. The mung beans were placed on a metal screen inside an enclosed stainless steel cylindrical chamber and hot air at 250° C. (heating zone) was blown in an upward direction through the bottom of the screen at a velocity sufficient to fluidize the mung beans (into air) to facilitate contact between the hot air and mung beans. The mung beans were exposed to hot air for anywhere between 20 second to 30 min. The hot air can also be blown from the top to bottom direction, relative to the metal screen bed, in other configurations of fluidized bed dryers. After the heat treatment was completed, the room temperature or cold air was blown onto the beans to cool down the mung beans to 30° C.−50° C. (cooling zone). After the beans were cooled to a desired temperature, the enclosed stainless steel chamber was opened and the beans beans were milled to prepare the heat treated flour.

The heat treated beans were milled in a hammer mill (Hosokawa Micron Powder Systems, 10 Chatham Rd, Summit, N.J. 07901). The mung beans were fed through a screw conveyor into the milling chamber. The chamber contains a rotating shaft with mounted swinging hammers to reduce the particle size of the mung beans to flour. The particle size was maintained using a screen in the mill to ensure no more than 10% of the particles were under a desired particle size. The flour was collected in a container and capped immediately after milling for further usage.

### Example 2: Volatile Compound Analysis of Roasted and Steam Treated Mung Bean Flour

The volatile small molecule compounds present in the heat treated mung bean flour of Example 1 were determined by head space gas analysis by GC/MS.

For extraction of the volatile small molecule compounds, mung bean flour (2 g) was placed in 20 mL GC glass headspace extraction vial (Size 22×75 mm, Restek, Bellefonte, Pa.). A polytetrafluoro ethylene septa and metal screw cap (Thread size 20 mm, Restek, Bellefonte, Pa.) was used to cap each vial and incubated for 60 minutes at 90° C. An extraction phase followed the 60-minute incubation period at 90° C., when volatile compounds were collected from the headspace (HS) of the vial. The volatile small molecule compounds present in the head space of the heat treated pulse flour were analyzed by GC/MS.

Thermo 1310 gas chromatograph (Thermo Fisher Scientific, Waltham, Mass.) with a Thermo TSQ8000 Evo mass spectrometer (Thermo Fisher Scientific, Waltham, Mass.) was used for analysis. A Thermo Scientific TriPlus RSH Autosampler was used to load vials in the agitator. After extraction was completed, volatile compounds were desorbed onto a HP-5MS capillary column (30 m×0.25 mm×0.25 μm; Agilent J&W GC Columns, Santa Clara, Calif.)—that resulted in the separation of the compounds. The compounds separated by the GC were then analyzed by mass spectrometery to detect ions within a range of 40-400 m/z with an electro mode at 70 eV. All volatile compound peaks were explored using Chromeleon 7.2 software (Thermo Fisher Scientific, Waltham, Mass.) and identified with National Institute of Standards and Technology (NIST) main library match (NIST, Gaithersburg, Md., USA) using match factor (SI) and reverse match factor (RSI) scores and probability percentages as parameters to select compounds with confidence. Match scores are parameters to interpret mass spectral match quality. The best candidates for volatile compound peaks were selected based on higher match scores (a match score of >600 is acceptable).

Table 1 shows the results of the amounts of volatile small molecule compounds, as measured by peak intensity counts of the GC trace of non heat treated mung bean flour (control), heat treated pulse flour without the use of steam and heat treated pulse flour that was treated with both heat and steam of Example 1.

TABLE 1

| | | Non-Heat Treated | Roasted Flour | Roasted and Steamed |
|---|---|---|---|---|
| | | Flour | Intensity, | flour |
| | | Peak Intensity | Peak Intensity | Peak Intensity |
| Compound | Flavor/Odor | Counts | Counts | Counts |
| heptane | Sweet, | 1.2 × 10⁷ | 9.5 × 10⁶ | 5.0 × 10⁶ |
| | alkane-like | | | |
| 3-methyl, 1-butanol | Alcoholic | 1.0 × 10⁶ | 4.0 × 10⁵ | 4.0 × 10⁵ |
| 4-methyl heptane | | 7.5 × 10⁶ | 7.0 × 10⁶ | 1.5 × 10⁶ |
| 1-pentanol | Grassy/Green | 9.0 × 10⁵ | 6.0 × 10⁵ | 4.5 × 10⁵ |
| 2,4-dimethylhept-1- | Alkene like | 2.6 × 10⁷ | 2.6 × 10⁷ | 4.0 × 10⁶ |
| ene | | | | |
| hexanal | Beany flavor | 3.2 × 10⁶ | 1.2 × 10⁶ | 1.0 × 10⁶ |
| benzoic acid, methyl | Fruity, | 2.0 × 10⁶ | 2.2 × 10⁶ | 1.5 × 10⁶ |
| ester | phenolic | | | |

Table 2 shows the percent reduction of volatile small molecule compounds in mung beans that were heat treated, without steam treatment as disclosed in Example 1 as compared to non-heat treated pulse flour.

TABLE 2

| | | Percent Reduction |
|---|---|---|
| Compound | Flavor Notes | from Roasting |
| 2-methyl, 1 pentanol | pungent, fermented, | 87 |
| | potential off-flavor | |
| 3-Trifluoroacetoxydodecane | potentially fruity | 52 |
| 2-Nonyn-1-ol | green and vegetative | 88 |
| 1-hexanol | beany, off-flavor | 29 |
| 2-butyl,1-Octanol | waxy, green and floral | 63 |
| 5-Tridecene | alkene-like | 83 |
| 2,3,5,8-tetramethyl-Decane | potentially fungus-like | 89 |
| 2-ethyl, 1-Decanol | waxy, green, fatty, floral/sweet | 56 |
| 4-Methyldocosane | waxy, herbal, lemon-scented | 52 |
| 3-Pentyl-2,4-Pentadien-1-ol | green, fruity, sweet | 74 |
| 2-Dodecenal | green, citrus/herbal, waxy | 79 |
| 1-chloro,Octadecane | green | 67 |
| Di-tert-dodecyl disulfide | sulfurous, roasty | 66 |

### Example 3: Ultrafiltration Process for Preparing Pulse Protein Isolates

Ultrafiltered Pulse Protein Isolate: 40 kg of Mung bean flour (102) that was preprocessed by drying and grinding was extracted (104) with 200 kg water, 600 g salt (NaCl), 100 mL antifoam in a Breddo liquefier (Corbion Inc). The mixing was performed for 2.5 minutes. The pH at the end of the run was adjusted to 7.0 using 1 M NaOH solution. The flour slurry (105) was then centrifuged to perform a starch solid separation (106) using a decanter (SG2-100, Alfalaval Inc). The major portion of the starch solids and unextracted material (decanter heavy phase) was separated from the liquid suspension (decanter light phase). The resuspension stream (light phase) was further clarified using a disc stack centrifuge (Clara 80, Alfalaval Inc.) into a high solids slurry (disc stack heavy phase) and a clarified resuspension (107—disc stack light phase). The disc stack heavy phase typically consists of fat, ash, starch and the protein carried over with the liquid portion of the slurry.

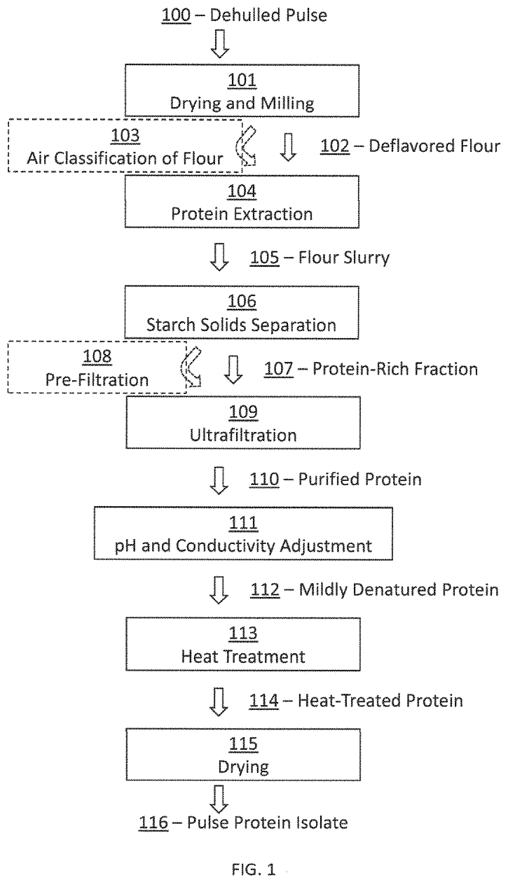

Half of the disc stack light phase (protein-rich fraction) was then processed through an ultrafiltration-diafiltration process (109) with a custom designed membrane purification unit (Alfalaval Inc.). This membrane unit was setup with a 10 kDa membrane from Alfalaval Inc. (3838RC10PP). The disc stack light phase was concentrated from 75 kg to about 20 kg (3-4 X concentration). The concentrated protein suspension was further diafiltered with DI water in three steps adding about equal amount of water at each step as the concentrate weight. The stream (110) of diafiltered UF concentrate (19.5 kg) was then collected and the pH of this concentrate was adjusted (111) from 7 to 6.1 using 20% w/w citric acid solution. Salt (NaCl) was added to adjust the conductivity in the 2-3 mS/cm range and not modified. The mildly denatured protein concentrate material (112) was then heat treated (113) using a microthermics UHT unit with the pasteurization condition set at 72.5° C. and 30 sec hold time. The heat-treated material (114) was then spray dried (115) with a SPX Anhydro M400 spray dryer (GEA Niro Inc.) with the inlet temp at 180° C., outlet temp at 85° C. using a nozzle atomizer to obtain protein isolate (116). An illustration of this process, including the numbers (102-116) noted above, is shown in FIG. 1.

Isoelectrically-Precipitated Pulse Protein Isolate Control: The other half of the disc stack light phase was then transferred to the liquefier tank. The pH was adjusted to 5.6 with 20% w/w citric acid. The slurry was mixed and run through the decanter (SG2-100, Alfalaval Inc.) in recirc mode until the spin down on the decanter light phase was negligible. Then the decanter was shut down and the protein pellet collected on the decanter heavy phase side. The pellet was resuspended with 3.5 X deionized water to get the concentration in the range to minimize spray drier losses. The resuspended protein solution was adjusted to a pH of 6 using 1M NaOH and salt was added to obtain the conductivity in the 2-3 mS/cm range. This material was then heat treated and spray dried to obtain an isoelectrically precipitated isolate for use as a control in Examples 3-6.

### Example 4: Volatile Compound Identification and Sensory Analysis of Roasted and Steam Treated Mung Bean Flour by GCMS-O

GCMS-O is an analytical technique that combines gas chromatography, mass spectroscopy and olfactometric detection detection of odorous substances (volatile organic compounds). Briefly, the technique is performed by GC/MS analysis, concurrently with a trained sensory evaluator evaluating the odor of the eluate by sniffing. This technique is well established scientifically and is available from fee-for-service laboratories. The GCMS-O analysis of this example was performed by Volatile Analysis Corportion.

The volatile small molecule compounds present in roasted and steam treated mung bean flour were determined by head space solid phase microextraction analysis by GCMS-O and odor contribution of individual VOCs were determined by comparing GCMS-O to sensory analysis of the flour.

Duplicate portions of each mung bean material (30 g) were transfer into separate 200 mL clear glass collection jars fitted with Teflon lined closures. The containers were then sealed and set aside for headspace equilibration at laboratory environment (25° C. and −30% RH).

To perform sensory analysis of the flour samples one sample jar from each set of duplicates was opened and the interior environment was subjected to thorough odor and aroma assessment by a trained sensory evaluator after 4 hours of headspace equilibration. At the same incubation timepoint samples were also prepared for GCMS-O analysis. Solid phase microextraction fibers (2 cm) coated with carboxen-PDMS were inserted through the pinholes in the closures of the collection jars and exposed to the interior environment for 18 hours for extraction of VOCs for GCMS-0 analysis.

After completion of each headspace extraction, the SPME fibers were immediately transferred to the inlet of a gas chromatograph (GC). The collected volatiles and semi-volatiles were thermally desorbed at 260° C. and passed to the analytical column for separation. The chromatographic system used for this analysis consisted of an Agilent 6890 Gas Chromatograph (GC) equipped with two capillary columns (1st column—30-meter low polarity capillary column; 2nd column—30-meter high polarity capillary column→serial column configuration), Agilent 5975 Mass Spectrometric Detector (MSD) and olfactory port/detector (sniff port/ODP, heated to 240° C.). During each chromatographic run, the effluent (after passing through both columns) was split between the sniff port and MSD. The trained scientist sensory evaluator sniffed the odorous molecules at the sniff port and the olfactory data were recorded in form of aromagrams, using proprietary AromaTrax™ software. The aromagrams are the graphical representation of sample's odor/intensity versus retention time in the GC-MS chromatogram format. Agilent MSD ChemStation® acquisition software was used to record the GC-MS traces (chromatograms). The aromagrams and corresponding chromatograms of each sample were acquired simultaneously.

The Agilent MSD ChemStation® Data Analysis program, in conjunction with NIST11, Wiley9, FFNSC13 MS Libraries, and Microanalytics™ proprietary aroma database, were used to analyze the GCMS data. All compound identifications provided are based on commercial MS library matches and/or internal databases.

The sensory scale below is a seven-point scale used for characterizing odor impact of individual VOCs on overall odor of the flour based on GCMS-0 analysis of individual volatile compounds and sensory analysis of the flour.

Table 3 shows a comparison of individual VOCs that display a reduced impact on overall odor of mung bean flour when mung beans are roasted, or roasted and steam treated. In table 3, C3-pyrazine refers to isomers of methyl, ethyl pyrazine, propyl pyrazine or isopropyl pyrazine and dimethyl pyrazine refers to 1,5-dimethyl pyrazine or 2,3-dimethyl pyrazine. One of skill in the art using well known analytical techniques can identify these isomers. Analytical techniques for identifying if C3-pyrazine is a particular isomer or a mixture of isomers or if dimethyl pyrazine is 1,5-dimethyl pyrazine, 2,3-dimethyl pyrazine or a mixture include GC, GC-MS, GC-MS-MS, GC-NMR, LC, LC-MS, LC-MS-MS, LC-NMR and other techniques.

TABLE 3

| | | | Mung bean flour | |
|---|---|---|---|---|
| | | | with roast | Mung bean flour with |
| Volatile Organic | Odor | Raw Mung | treatment | roasted and stream |
| Compounds | Character | Bean Flour | condition | treated condition |
| Identification | descriptors | (Odor Impact) | (Odor Impact) | (Odor Impact) |
| Diacetyl (butane- | Buttery, | high | high | moderate high |
| 2,3-dione) | sweet | | | |
| 1-butanol + | Oily, musty, | moderate high | moderate | moderate low |
| 2,3-pentanedione | buttery, | | | |
| | aldehydic | | | |
| 1-pentanol | Sweet, fruity, | moderate | moderate | moderate low |
| | sharp | | | |
| hexanal | Grassy, | dominant | very high | high |
| | green, sharp | | | |
| acetic acid | Acidic, sour | very high | high | high |
| 2-hexenal | Oily, | moderate high | moderate high | moderate low |
| | aldehydic, | | | |
| | fruity, sweet | | | |
| 2-butylfuran | sharp, beans, | moderate | ND | low |
| | green | | | |
| 1-hexanol | Sharp, | very high | high | moderate high |
| | almond, | | | |
| | solvent | | | |
| heptanal + 2- | musty, oily, | high | moderate | moderate high |
| heptanol | sweet, | | | |
| | solvent | | | |
| 2-heptenal | Musty, | moderate high | high | ND¹ |
| | rancid, oily | | | |
| dimethyl pyrazine | nutty, roasted | moderate | moderate low | moderate |
| isomer | | | | |
| 2-amylfuran + | sweet, | high | ND¹ | ND¹ |
| γ-butyrolactone | nauseating, | | | |
| | musty, sharp | | | |
| d, l-limonene + | sweet, fruity, | high | high | moderate |
| phellandrene ((α): | herbaceous | | | |
| 2-Methyl-5- | | | | |
| (propan-2- | | | | |
| yl)cyclohexa-1,3- | | | | |
| diene | | | | |
| (β): 3-Methylidene- | | | | |
| 6-(propan-2- | | | | |
| yl)cyclohex-1-ene) | | | | |
| 3-octene-2-one + | roasted, | High | High | ND¹ |
| C3-pyrazine | sweet, musty, | | | |
| | oily | | | |
| 2-octenal | woody, | moderate | moderate high | ND¹ |
| | earthy, stale, | | | |
| | oily | | | |
| nonanal | aldehydic, | high | moderate | moderate |
| | oily | | | |
| benzyl alcohol | oily, musty, | high | high | ND¹ |
| | solvent, | | | |
| | sharp | | | |
| phenethyl alcohol | floral, sweet, | high | high | ND¹ |
| | fresh, green | | | |
| trans-2-nonanal | aldehydic, | moderate high | moderate | moderate |
| | cardboard, | | | |
| | paper | | | |
| β-ionone | sweet, floral, | moderate high | moderate | moderate |
| | sharp | | | |
| undecanal | aldehydic, | moderate | moderate low | ND¹ |
| | oily, fruity | | | |
| methyl eugenol | roasted, | moderate low | ND¹ | ND¹ |
| | sweet, smoky | | | |

ND—Odor not detected or below “low” odor score

### Example 5: Volatile Compound Analysis of Roasted and Steam Treated Mung Bean Flour by ITEX-DHS GCMS

A GC-MS system—Thermo 1310 gas chromatograph (Thermo Fisher Scientific, Waltham, Mass.) with a Thermo TSQ8000 Evo mass spectrometer (Thermo Fisher Scientific, Waltham, Mass.) was used for analysis. For extraction of volatiles, mung bean flour (2 g) was placed in 20 mL GC glass headspace extraction vial (Size 22×75 mm, Restek, Bellefonte, Pa.) and spiked with 0.5 ug internal standard 1,2-dichlorobenzene-d4 (Millipore Sigma, Burlington, Mass.). A polytetrafluoro ethylene septa and metal screw cap (Thread size 20 mm, Restek, Bellefonte, Pa.) was used to cap each vial. A Thermo Scientific TriPlus RSH Autosampler was used to load vials in the agitator. After a 15-minute incubation period at 80° C., VOCs were extracted from the sample by dynamic headspace (DHS) with 25 extraction strokes. Gas (helium) flow rate used was 1 mL/min and a target column temperature of 220° C. was applied. The DHS technique utilized a PAL3 ITEX Trap Tenax TA 80/100 mesh (23 needle gauge size, LEAP PAL Parts and Consumables, Raleigh, N.C.) for extraction. After extraction was completed, volatile compounds were desorbed onto a HP-5MS capillary column (30 m×0.25 mm×0.25 μm; Agilent J&W GC Columns, Santa Clara, Calif.)—that resulted in the separation of the compounds. A mass spectrometer was used to detect ions within a range of 40-400 m/z with an electro mode at 70 eV.

All volatile compound peaks were analyzed using Chromeleon 7.2 software (Thermo Fisher Scientific, Waltham, Mass.). VOCs were identified either by use of reference standards or NIST mass spectra library match. For reference standard confirmed VOCs, high purity (>98%) analytical reference standard runs were used to confirm VOC identification by corroborating retention time and mass spectra with those of compounds in the flour samples. For identification by NIST library match, match factor (SI) and reverse match factor (RSI) scores using the normal algorithm were used. A match score of >800 was considered acceptable for identification of a compound.

Peak area quantitation of each individual volatile organic compound component was quantitated by normalizing the raw peak area data by internal standard 1,2-dichlorobeneze-d4 (Millipore Sigma, Burlington, Mass.) response.

Table 4 shows a comparison of the relative abundance of VOCs in raw and heat treated mung bean flours as determined by internal standard normalized peak areas from the chromatogram. The table includes all identified compounds observed to be decreased or increased in the treated mung bean flours. The peak areas were normalized to internal standard, 1,2-dichlorobenzene-d4 (IS). Table 4 shows that compounds that either decreased or increased as mung bean flour was heat treated and/or roasted and steam treated.

TABLE 4

| | | | | Mung bean flour |
|---|---|---|---|---|
| | | | Mung bean flour | with roasted |
| | | Raw Mung | with roast treatment | and steam |
| | | Bean Flour | condition | treated condition |
| | | (IS | (IS | (IS |
| Volatile Organic | | normalized | normalized | normalized |
| Compounds | Odor descriptors | peak area) | peak area) | peak area) |
| 1-Hexanol¹ | sharp, almond, solvent, | 1.77 × 10⁷ | 9.52 × 10⁶ | 1.00 × 10⁷ |
| | flower, green, resin | | | |
| Hexanoic acid methyl | fruity type odor and | 3.66 × 10⁶ | 6.63 × 10⁵ | 7.83 × 10⁴ |
| ester¹ | flavor, sweet | | | |
| 2-pentyl furan¹ | sweet, nauseating, musty, | 1.63 × 10⁶ | 1.53 × 10⁶ | 1.27 × 10⁶ |
| | sharp | | | |
| 3-Penten-2-one¹ | fruity type odor and/or | 4.77 × 10⁶ | 1.49 × 10⁶ | 1.53 × 10⁶ |
| | fishy type flavor | | | |
| Butanoic acid methyl | characteristic sweet and | 1.53 × 10⁵ | 1.32 × 10⁵ | 1.20 × 10⁵ |
| ester² | fruity odor | | | |
| 2-Heptenal¹ | musty, rancid, oily | 3.18 × 10⁴ | — | — |
| Heptanal + 2-Heptanol¹ | musty, aldehydic, oily, | 3.58 × 10⁵ | 2.29 × 10⁵ | 2.27 × 10⁵ |
| | sweet, solvent | | | |
| 13,16-Octadecadiynoic | oily, fatty, woody | 3.11 × 10⁴ | — | — |
| acid, methyl ester² | | | | |
| Tetradecene² | oil-like | 1.01 × 10⁵ | 7.33 × 10⁴ | 7.12 × 10⁴ |
| Decane² | tasteless | 1.15 × 10⁵ | 1.15 × 10⁵ | 8.49 × 10⁴ |
| 3-carene² | sweet, pungent odor, fir | 3.01 × 10⁵ | 4.58 × 10⁵ | 3.31 × 10⁵ |
| | needles, musky earth and | | | |
| | damp woodlands | | | |
| | combination | | | |
| Dodecane² | gasoline-like to odorless, | 2.99 × 10⁵ | 4.85 × 10⁵ | 3.18 × 10⁵ |
| | mild unpleasant odor | | | |

¹VOCs confirmed by analytical reference standard runs and validated by retention time, mass spectra, and library match analyses.
²VOCs confirmed by NIST Library normal algorithm with mass spectra match factors and reverse match factors >800
— below limit of quantitation

Tables 1, 2, 3 & 4 shows the relative abundance of VOCs in raw and heat treated mung bean flours as determined by internal standard normalized peak areas from chromatograms. The VOCs present in Mung bean flour prepared from roasted mung beans and steam roasted mung beans decreased, increased or remained the same as compared to the VOCs present in mung bean flour prepared from mung beans that were not heat treated. As one example from Table 2, the amount of hexanoic acid methyl ester present in roasted mung bean flour was reduced by greater than 80% as compared to unroasted mung bean flour. Similarly, the amount of 1-Hexanol present in roasted mung bean flour was reduced by about 50% as compared to unroasted mung bean flour. Heat treatment reduced 2-heptanal and 13,16-Octadecadiynoic acid, methyl ester to undetectable levels

Tables 1 and 4 shows that three compounds: 3-carene, decane and dodecane increased upon heat treatment without steam or heat treatment in the presence of steam.

The present disclosure is not to be limited in scope by the specific embodiments described herein. Indeed, various modifications of the invention in addition to those described herein will become apparent to those skilled in the art from the foregoing description. Such modifications are intended to fall within the scope of the appended claims.

What is claimed is:

1. A heat treated pulse flour prepared by heat treating a pulse, in the presence or absence of steam, and milling the pulse to prepare the heat treated pulse flour, the heat treated pulse flour comprising volatile small molecule compounds, wherein the amount of volatile small molecule compounds present in the heat treated pulse flour is decreased, increased or is unchanged as compared to the amount of volatile small molecule compounds present in a pulse flour that is not heat treated, and wherein the amount of at least one volatile small molecule compound is increased or decreased.

2. The heat treated pulse flour of claim 1, wherein the pulse is heat treated by contacting the pulse with steam.

3. The heat treated pulse flour of claim 1, wherein the pulse is heat treated without exposure to steam.

4. The heat treated pulse flour of claim 2, wherein the pulse is heat treated with exposure to steam, the steam at a temperature of between 100° C. to 500° C.

5. The heat treated pulse of any one of claims 1-4, wherein the pulse selected from the group consisting of dry beans, lentils, mung beans, fava beans, dry peas, chickpeas, cowpeas, bambara beans, pigeon peas, lupins, vetches, adzuki, common beans, fenugreek, long beans, lima beans, runner beans, tepary beans, soy beans, mucuna beans, and combinations thereof.

6. The heat treated pulse flour of any of claims 1-5, wherein the pulse is dehulled.

7. The heat treated pulse flour of any one of claims 5-6, wherein the pulse is of the genus Vigna.

8. The heat treated pulse flour of claim 7, wherein the pulse is Vigna angularis or Vigna radiata.

9. The heat treated pulse flour of claim 8, wherein the pulse is Vigna radiata.

10. The heat treated pulse flour of any one of claims 1-9, wherein the volatile small molecule compound is selected from the group consisting of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; 0-ionone; undecanal; methyleugenol; 3-carene; dodecane; and combinations thereof.

11. The heat treated pulse flour of any one of claims 1-10, wherein the amount of volatile small molecule compounds present in the heat treated pulse flour is decreased as compared to the amount of volatile small molecule compounds present in a pulse flour that is not heat treated.

12. The heat treated pulse flour of claim 11, wherein the volatile small molecule compound is selected from the group consisting of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; 0-ionone; undecanal; methyleugenol; and combinations thereof.

13. The heat treated pulse flour of any one of claims 1-12, wherein the amount of volatile small molecule compounds present in the heat treated pulse flour is increased as compared to the amount of small molecule compounds present in a pulse flour that is not heat treated.

14. The heat treated pulse flour of claim 13, wherein the volatile small molecule compound is selected from the group consisting of 3-carene and dodecane.

15. The heat treated pulse flour of any one of claims 1-10, wherein the amount of volatile small molecule compounds present in the heat treated pulse flour is unchanged as compared to the amount of small molecule compounds present in a pulse flour that is not heat treated.

16. The heat treated pulse flour of claim 13, wherein the volatile small molecule compound is selected from the group consisting of 2,4-dimethylhept-1-ene; decane and benzoic acid, methyl ester.

17. The heat treated pulse flour of any one of claims 1-16, wherein the amount of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; β-ionone; undecanal; or methyleugenol is decreased while concurrently the amount of 3-carene or dodecane is increased and/or concurrently the amount of 2,4-dimethylhept-1-ene; decane or benzoic acid; methyl ester; or a combination thereof remains the same compared to pulse flour that has not been heat treated.

18. The heat treated pulse flour of any one of claims 1-14, wherein the amount of volatile small molecule compounds are determined by analyzing the volatile small molecule compounds obtained by headspace gas analysis, in-tube extraction dynamic headspace (ITEX-DHS), stirbar sorptive extraction (SBSE), solid phase microextraction (SPME), or purge and trap.

19. An isolated protein obtained from a heat treated pulse flour, the heat treated pulse flour prepared by heat treating a pulse, in the presence or absence of steam and milling the pulse to prepare the heat treated pulse flour, the heat treated pulse flour comprising volatile small molecule compounds, wherein the amount of volatile small molecule compounds present in the heat treated pulse flour is decreased, increased, or is unchanged as compared to the amount of small molecule compounds present in a pulse flour that is not heat treated, and wherein the amount of at least one volatile small molecule compound is increased or decreased.

20. An isolated protein obtained from a heat treated pulse flour of claim 19, wherein the pulse is heat treated by contacting the pulse with steam.

21. The isolated protein of claim 20, wherein the pulse is heat treated without exposure to steam.

22. The isolated protein flour of claim 20, wherein the pulse is treated with the exposure to steam, the steam at a temperature of between 100° C. to 500° C.

23. The isolated protein of any one of claims 19-22, wherein the pulse selected from the group consisting of dry beans, lentils, mung beans, fava beans, dry peas, chickpeas, cowpeas, bambara beans, pigeon peas, lupins, vetches, adzuki, common beans, fenugreek, long beans, lima beans, runner beans, tepary beans, soy beans, and mucuna beans.

24. The isolated protein of any of claims 19-23, wherein the pulse is dehulled.

25. The isolated protein of claim 24, wherein the pulse is of the genus Vigna.

26. The isolated protein of claim 25, wherein the pulse is Vigna angularis or Vigna radiata.

27. The isolated protein of claim 26, wherein the pulse is Vigna radiata.

28. The isolated protein of any one of claims 19-27, wherein the small molecule is selected from the group consisting of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; β-ionone; undecanal; methyleugenol, 3-carene; dodecane; and combinations thereof.

29. The isolated protein of any one of claims 19-28, wherein the amount of volatile small molecule compounds present in the heat treated pulse flour is decreased as compared to the amount of small molecule compounds present in a pulse flour that is not heat treated.

30. The isolated protein of claim 29, wherein the volatile small molecule compound is selected from the group consisting of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; or di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; 0-ionone; undecanal; methyleugenol; and combinations thereof.

31. The isolated protein of any one of claims 19-30 wherein the amount of volatile small molecule compounds present in the heat treated pulse flour is increased as compared to the amount of volatile small molecule compounds present in a pulse flour that is not heat treated.

32. The isolated protein of claim 31, wherein the volatile small molecule compound is selected from the group consisting of 3-carene and dodecane.

33. The isolated protein of any one of claims 19-30, wherein the amount of volatile small molecule compounds present in the isolated protein is unchanged as compared to the amount of small molecule compounds present in an isolated protein obtained from a pulse flour that is not heat treated.

34. The isolated protein of claim 33, wherein the volatile small molecule compound is selected from the group consisting of 2,4-dimethylhept-1-ene; decane and benzoic acid, methyl ester.

35. The isolated protein of any one of claims 19-34, wherein the amount of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; f3-ionone; undecanal; or methyleugenol is decreased while concurrently the amount of 3-carene or dodecane is increased and/or concurrently the amount of 2,4-dimethylhept-1-ene; decane or benzoic acid; methyl ester; or a combination thereof remains the same compared to isolated protein obtained from pulse flour that is not heat treated.

36. The isolated protein of any one of claims 19-35, wherein the amount of volatile small molecule compounds are determined by analyzing the volatile small molecule.

37. The isolated protein of claim 32, wherein the amount of volatile small molecule compounds are determined by analyzing the volatile small molecule compounds obtained by headspace gas analysis, in-tube extraction dynamic headspace (ITEX-DHS), stirbar sorptive extraction (SBSE), solid phase microextraction (SPME), or purge and trap.

38. An egg substitute comprising the isolated protein of any one of claims 19-37.

39. A method of manufacturing a heat treated pulse flour, the method comprising the steps of:
exposing the pulse, in the presence or absence of steam, to one or more heating zones for a desired amount of time to prepare a heat treated pulse;
wherein the temperature of one heating zone is different than the temperature of another heating zone;
optionally, exposing the pulse to a cooling zone to cool the heat treated pulse to a desired temperature; and
milling the heat treated pulse to prepare the heat treated pulse flour;
wherein the heat treated pulse flour comprises volatile small molecule compounds, and wherein the amount of volatile small molecule compounds present in the heat treated pulse flour is increased or decreased or unchanged as compared to the amount of small molecule compounds present in a pulse flour that is not heat treated; and wherein the amount of at least one volatile small molecule compound is increased or decreased.

40. The method of claim 39, wherein the method comprises exposing the pulse to at least two heating zones.

41. The method of any of claim 39 or 40, wherein the pulse is heat treated by contacting the pulse with steam.

42. The method of any one of claims 39-41, wherein the steam is at a temperature of between 100° C. to 500° C.

43. The method of any one of claims 39-42, wherein the temperature of the one or more heating zones is between 75° C. to 500° C.

44. The method of any one of claim 39-43, wherein the temperature of a first heating zone is lower than the temperature of a second heating zone.

45. The method of any one of claims 39-44, wherein the temperature of a first heating zone is between 100° C. to 150° C.

46. The method of any one of claims 39-45, wherein the temperature of a second heating zone is between 175° C. to 225° C.

47. The method of any one of claims 39-46, wherein the residence time of the pulse in the one or more heating zones is between 5 seconds and 30 minutes.

48. The method of claim 47, wherein the residence time of the pulse in the one or more heating zones is between 1 minute and 5 minutes.

49. The method of any one of claims 39-48, wherein the temperature of the cooling zone is between 10° C. to 50° C.

50. The method of any one of claims 39-49, wherein the residence time of the pulse in the cooling zone is between 1 minute and 60 minutes.

51. The method of any one of claims 39-50, wherein the pulse selected from the group consisting of dry beans, lentils, mung beans, fava beans, dry peas, chickpeas, cowpeas, bambara beans, pigeon peas, lupins, vetches, adzuki, common beans, fenugreek, long beans, lima beans, runner beans, tepary beans, soy beans, and mucuna beans.

52. The method of claim 51, wherein the pulse is of the genus Vigna.

53. The method of claim 52, wherein the pulse is Vigna angularis or Vigna radiata.

54. The method of claim 53, wherein the pulse is Vigna radiata.

55. The method of any one of claims 51-54, wherein the small molecule is selected from the group consisting of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; β-ionone; undecanal; methyleugenol; 3-carene and dodecane; and combinations thereof.

56. The method of any one of claims 51-55, wherein the amount of volatile small molecule compounds present in the heat treated pulse flour is decreased as compared to the amount of volatile small molecule compounds present in a pulse flour that is not heat treated.

57. The method of claim 56, wherein the volatile small molecule compound is selected from the group consisting of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; β-ionone; undecanal; methyleugenol; and combinations thereof.

58. The method of any one of claims 51-55, wherein the amount of volatile small molecule compounds present in the heat treated pulse flour is increased as compared to the amount of volatile small molecule compounds present in a pulse flour that is not heat treated.

59. The method of claim 58, wherein the volatile small molecule compound is selected from the group consisting of 3-carene and dodecane.

60. The method of any one of claims 39-55, wherein the amount of volatile small molecule compounds present in the heat treated pulse flour is unchanged as compared to the amount of small molecule compounds present in a pulse flour that is not heat treated.

61. The method of claim 60, wherein the volatile small molecule compound is selected from the group consisting of 2,4-dimethylhept-1-ene; decane and benzoic acid, methyl ester.

62. The method of any one of claims 39-61, wherein the amount of heptane; 3-methyl, 1-butanol; 4-methyl heptane; 1-pentanol; 2,4-dimethylhept-1-ene; hexanal; benzoic acid, methyl ester; decane; 2-methyl; 1 pentanol; 3-trifluoroacetoxydodecane; 2-nonyn-1-ol; 1-hexanol; 2-butyl, 1-octanol; 5-tridecene; 2,3,5,8-tetramethyl-decane; 2-ethyl, 1-decanol; 4-methyldocosane; 3-pentyl-2,4-pentadien-1-ol; 2-dodecenal; 1-chloro, octadecane; di-tert-dodecyl disulfide; diacetyl (butane-2,3-dione); 2,3-pentanedione; acetic acid; 2-hexenal; 2-butylfuran; heptanal; 2-heptanol; 2-heptenal; dimethyl pyrazine; 2-amylfuran; γ-butyrolactone; d,l-limonene; phellandrene; 3-octene-2-one; C3-pyrazine; 2-2-octenal; nonanal; benzyl alcohol; phenethyl alcohol; trans-2-nonanal; f3-ionone; undecanal; or methyleugenol is decreased while concurrently the amount of 3-carene or dodecane is increased and/or concurrently the amount of 2,4-dimethylhept-1-ene; decane or benzoic acid; methyl ester; or a combination thereof remains the same compared to pulse flour that has not been heat treated.

63. The method of any one of claims 39-59, wherein the amount of volatile small molecule compounds are determined by analyzing the volatile small molecule compounds obtained by headspace gas analysis, in-tube extraction dynamic headspace (ITEX-DHS), stirbar sorptive extraction (SBSE), solid phase microextraction (SPME), or purge and trap.

64. A method of preparing a pulse protein isolate, the method compromising the steps of:
obtaining a heat treated pulse flour of any one of claims 39-63; and
isolating the pulse proteins by ultrafiltration or isoelectric precipitation.

65. The method of claim 64, wherein ultrafiltration is performed by:
extracting protein from the milled composition comprising pulse proteins in an aqueous solution at a pH of from about 1 to about 9 to produce a protein rich fraction containing extracted pulse proteins;
applying the protein rich fraction to an ultrafiltration process comprising a semi-permeable membrane to separate a retentate fraction from a permeate fraction based on molecular size at a temperature of from 2° C. to 60° C.; and
collecting the retentate fraction containing the pulse protein isolate.
